(19) United States
(12) Patent Application Publication
(10) Pub. No.: US 2005/0287213 A1
(43) Pub. Date: Dec. 29, 2005

(54) Dosage forms for low solubility and or low dissolution rate free acid pharmaceutical agents

(76) Inventors: Patrick S.L. Wong, Burlingame, CA (US); Noymi V. Yam, Sunnyvale, CA (US); Sherry Xiuling Li, Cupertino, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/148,679

(22) Filed: Jun. 8, 2005

Related U.S. Application Data
(60) Provisional application No. 60/583,701, filed on Jun. 28, 2004.

(51) Int. Cl. A61K 9/64; A61K 9/24

(52) U.S. Cl. 424/473

(57) ABSTRACT

An osmotic controlled release dosage form is described comprising a core comprising a first drug composition, wherein the first drug composition comprises topiramate and/or its pharmaceutically acceptable salt; a semi-permeable wall surrounding the core; and an exit orifice through the semi-permeable wall for releasing the first drug composition from the dosage form over a prolonged period of time.

### CROSS-REFERENCE TO RELATED APPLICATION

This application claims benefit, under 35 U.S.C 119(e), of U.S. Ser. No. 60/583,701, filed Jun. 28, 2004, which is incorporated herein by reference.

### FIELD OF THE INVENTION

The present invention is directed to dosage forms containing low solubility and/or low dissolution rate free acid pharmaceutical agents, methods for the preparation of such dosage forms, and methods of treatment comprising administering, to a subject in need thereof, the dosage forms of the present invention.

### BACKGROUND OF THE INVENTION

The art is replete with descriptions of dosage forms for sustained or controlled release of pharmaceutical agents. While a variety of sustained release dosage forms for delivering certain drugs may be known, not every drug may be suitably delivered from those dosage forms because of solubility, dissolution rate, metabolic processes, absorption and/or other physical, chemical and physiological parameters that are unique to the drug and/or the mode of delivery.

Dosage forms that incorporate free acid pharmaceutical agents characterized as having low solubility and/or low dissolution rates, including high drug loading dosage forms, provide a major challenge for controlled release delivery technology as these systems tend to result in tablets or capsules of such large size that patients are unwilling or unable to swallow them.

Free acid pharmaceutical agents characterized as having low solubility and/or low dissolution rates can be administered in multiple divided dosage forms, particularly at high dosage levels, for example at greater than or equal to about 100 mg/day. Thus conventional dosage forms of said low solubility and/or low dissolution rate free acid pharmaceutical agents may not lend themselves to controlled or sustained therapy, particularly for once-a-day administration.

One method to improve solubility of low solubility and/or low dissolution rate free acid pharmaceutical agents is to include solubilizing agents in the dosage form. It is well known that solubilizing agents, more particularly surfactants, can be used in liquid drug delivery systems as wetting agents, drug solubilizers, meltable carriers, oily liquid fills in gel capsules for oral administration, parenteral liquids for injection, ophthalmic drops, topical ointments, salves, lotions, and creams, suppositories, and in pulmonary and nasal sprays. By their amphipathic molecular structure comprising opposing polar hydrophilic and non-polar hydrophobic moieties with opposite physical and chemical properties, surfactants are well known to have poor cohesive properties. Accordingly, surfactants have been limited to the above applications because at room temperature, such surfactants are in the physical form of liquids, pastes, or brittle solids, which physical forms and properties are generally unacceptable for use as components in compressed solid tablets sufficiently durable for manufacture and practical use.

U.S. Pat. No. 6,569,463 describes using drug formulations consisting of coated granules, in which the coating consists of at least one surfactant and preferably a mixture of the surfactant with a hydrophobic drug and a lipophilic additive. This substrate coating facilitates rapid dispersion and provides rapid, sustained solubilization of the drug in the absence of liquid ingredients. The lipophilic additive further enhances solubilization of the drug or promotes dispersion in vivo.

As noted, surfactants typically have poor cohesive properties and therefore do not compress as hard, durable tablets. Furthermore, surfactants are in the physical form of liquid, pastes, or waxy solids at standard temperatures and conditions and are inappropriate for tabulated oral pharmaceutical dosage forms, such as might be used to deliver low solubility and/or low dissolution rate free acid pharmaceutical agents.

An additional concern with the use of surfactants to improve solubility and/or dissolution rate of low solubility and/or low dissolution rate free acid pharmaceutical agents is that addition of surfactant increases the amount of excipients in the dosage form. For dosage forms that have a high dose of drug, such an increase in excipients leads to a significant increase in the size of the dosage form. Such large dosage forms are infeasible and inconvenient for a patient to swallow.

Thus, there remains a need for a means to deliver low solubility and/or low dissolution rate free acid pharmaceutical agents, for example topiramate, particularly at high dosage levels, with various delivery patterns, in dosage forms that are feasible and convenient for patients to swallow.

More particularly, there remains a need for dosage forms that provide dose-regulated, preferably controlled release, therapy over a prolonged period of time with low solubility and/or low dissolution rate free acid pharmaceutical agents. There is also a need for effective dosing methods, dosage forms and devices that will permit the controlled release of low solubility and/or low dissolution rate free acid pharmaceutical agents over a prolonged period of time in order to increase the time between dosing, preferably to obtain a twice-a-day dosing regimen and most preferably to obtain a once-a-day dosing regimen. Such dosage forms should also have the capability of being formulated to deliver the drug composition in a substantially zero order rate of release, a substantially ascending rate of release, or in other hybrid release rates, as appropriate.

What is needed are compositions and methods that address the problems noted above.

### SUMMARY OF THE INVENTION

In an aspect, the invention relates to an osmotic controlled release dosage form comprising a drug composition comprising: a low solubility and/or low dissolution rate free acid pharmaceutical agent, and a pharmaceutically acceptable salt thereof.

In another aspect, the invention relates to an osmotic controlled release dosage form comprising a core comprising a first drug composition, wherein the first drug composition comprises a low solubility and/or low dissolution rate free acid pharmaceutical agent and/or its pharmaceutically acceptable salt; a semi-permeable wall surrounding the core; and an exit orifice through the semi-permeable wall for releasing the first drug composition from the dosage form over a prolonged period of time.

In yet another aspect, the invention relates to an osmotic controlled release dosage form comprising: a core comprising a first drug composition, a second drug composition and a push layer, wherein the first and second drug composition each comprise a low solubility and/or low dissolution rate free acid pharmaceutical agent and/or its pharmaceutically acceptable salt; a semi-permeable wall surrounding the core; and an exit orifice through the semi-permeable wall for releasing the first and second drug compositions from the dosage form over a prolonged period of time.

### BRIEF DESCRIPTION OF THE FIGURES

The following figures are not drawn to scale, and are set forth to illustrate various embodiments of the invention.

FIG. 1 illustrates an embodiment of an osmotic dosage form of the present invention, illustrating the dosage form prior to administration to a subject.

FIG. 2 illustrates the dosage form of FIG. 1 in opened section, illustrating a single internally housed drug composition.

FIG. 3 illustrates the dosage form of FIG. 1 in opened section view, illustrating a bi-layer comprising a drug composition and a separate and contacting push layer for pushing the drug composition from the dosage form.

FIG. 4 illustrates the dosage form of FIG. 1, which further comprising an immediate release external overcoat on the dosage form.

FIG. 5 illustrates an opened view of another embodiment of the dosage form of the present invention illustrating a tri-layer arrangement comprising two drug compositions in parallel arrangement and a separate and contacting push layer for pushing the drug layers from the capsule shaped dosage form.

FIG. 6 shows the results from the experiment described in Example 2

FIG. 7 shows the results from the experiment described in Example 4.

In the drawing figures and specification, like parts in related figures are identified by like numbers. The terms appearing earlier in the specification and in the description of the drawing figures, as well as embodiments thereof, are further described elsewhere in the disclosure.

### DETAILED DESCRIPTION OF THE INVENTION

The present invention is best understood by reference to the following definitions, the drawings and exemplary disclosure provided herein. All documents cited to herein are incorporated by reference as if reproduced fully herein.

The inventors have unexpectedly discovered that addition of pharmaceutically acceptable salts of low solubility and/or low dissolution rate free acid pharmaceutical agents to dosage forms, preferably osmotic controlled release dosage forms, comprising those low solubility and/or low dissolution rate free acid pharmaceutical agents provides an increase in dissolution of the low solubility and/or low dissolution rate free acid pharmaceutical agents. In this way, use of surfactants to increase solubility or dissolution rate can be reduced or eliminated. In an embodiment of the present invention, the inventive drug compositions and osmotic controlled release dosage forms are substantially free from solubilizing agents. In another embodiment of the present invention, the inventive drug compositions and osmotic controlled release dosage forms contain an amount of solubilizing agent insufficient to completely solubilize the low solubility and/or low dissolution rate free acid pharmaceutical agents. While not wishing to be bound by any particular mechanism, the inventors hypothesize that the more highly soluble pharmaceutically acceptable salts of such low solubility and/or low dissolution rate free acid pharmaceutical agents act to break up multi-molecular complexes or structures of the low solubility and/or low dissolution rate free acid pharmaceutical agent. This “dispersive” effect may serve to increase the effective solubility or dissolution rate of the recited low solubility and/or low dissolution rate free acid pharmaceutical agents.

The expressions “exit” and “exit orifice” shall mean an opening in a dosage form, which permits drug to exit the dosage form. Suitable examples include, but are not limited to, a passageway; an aperture; an orifice; and a bore. The expressions also include an orifice that is formed or formable from a substance or polymer that erodes, dissolves or is leached from the outer wall to thereby form an exit orifice.

By “dosage form” is meant a pharmaceutical composition or device capable of delivering a pharmaceutical agent. Suitable examples of dosage forms include, but are not limited to tablets, capsules, gel-caps, matrix forms, osmotic forms, immediate release forms, controlled release forms, sustained release forms, extended release forms, and the like. A preferred embodiment of the inventive dosage form is an osmotic controlled release dosage form.

As used herein, unless otherwise noted, the term “push layer” shall mean a formulation which does not contain pharmaceutical agent and which comprises an osmopolymer. Preferably, the push layer comprises and an osmopolymer and an osmoagent. The push layer may further optionally contain one or more inactive ingredients, for example disintegrants, binders, diluents, lubricants, stabilizers, antioxidants, osmotic agents, colorants, plasticizers, coatings and the like.

As used herein, unless otherwise noted, the terms “drug composition” shall mean a formulation comprising at least one low solubility and/or low dissolution rate free acid pharmaceutical agent. Preferably, the drug composition comprises a low solubility and/or low dissolution rate free acid pharmaceutical agent and/or its pharmaceutically acceptable salt. More preferably, the drug composition comprises a low solubility and/or low dissolution rate free acid pharmaceutical agent and/or its pharmaceutically acceptable salt and a structural polymer. The drug composition may further optionally contain one or more inactive ingredients, i.e., pharmaceutically acceptable excipients such as disintegrants, binders, diluents, lubricants, stabilizers, antioxidants, osmotic agents, colorants, plasticizers, coatings and the like.

As used herein, unless otherwise noted, the terms “pharmaceutical agent” and “drug” shall mean a pharmaceutical agent, drug, compound, prodrug or derivative thereof, and combinations thereof.

As used herein, unless otherwise noted, the terms “low solubility and/or low dissolution rate free acid pharmaceutical agent” and “low solubility and/or low dissolution rate free acid drug” shall mean a pharmaceutical agent or drug, which in each case is a free acid, and exhibits low solubility and/or low dissolution rate characteristics.

Suitable examples of low solubility and/or low dissolution rate free acid pharmaceutical agents include, but are not limited to acyclovir, aspirin, azathioprine, cefoxitin, furosemide, ganciclovir, glipizide, ibuprofen, ketoprofen, mefenamic acid, methotrexate, omeprazole, phenobarbitol, topiramate, valproic acid, and the like and combinations thereof. In a preferred embodiment, the low solubility and/or low dissolution rate free acid pharmaceutical agents is topiramate.

As used herein, unless otherwise noted, the term “pharmaceutically acceptable salt”, shall mean any salt of a low solubility and/or low dissolution rate free acid pharmaceutical agent whose cation does not contribute significantly to the toxicity or pharmacological activity of the salt, and, as such, they are the pharmacological equivalents of the low solubility and/or low dissolution rate free acid pharmaceutical agent. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base.

Representative bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

As used herein the term “low solubility” shall mean that the neat pharmaceutical agent (in the absence of surfactants or other excipients) exhibits a solubility of less than about 100 mg/ml in de-ionized water at 37° C. Preferably, low solubility shall mean a solubility of less than about 50 mg/ml, more preferably, less than about 25 mg/ml, more preferably still, less than about 15 mg/ml, more preferably still, less than about 10 mg/ml, more preferably still, less than about 5 mg/ml, most preferably, less than about 1 mg/ml.

As defined herein, the solubility of a pharmaceutical agent is determined by adding the pharmaceutical agent to stirred or agitated de-ionized water maintained in a constant temperature bath at a temperature of 37° C. until no more pharmaceutical agent dissolves. The resulting solution saturated with the pharmaceutical agent is then filtered, typically under pressure through a 0.8-micron Millipore filter, and the concentration of the pharmaceutical agent in the solution is measured by any appropriate analytical method including gravimetric, ultraviolet spectrophometry, chromatography, and the like. The solubility of the pharmaceutical agent is measured at equilibrium.

As used herein, the term “low dissolution rate” shall mean that rate of dissolution of the pharmaceutical agent under constant surface area (i.e the rate at which the pharmaceutical agent dissolves in de-ionized water at 37° C.) is between 0 mg/min/cm² and about 20 mg/min/cm², preferably, between about 0.1 mg/min/cm² and about 10 mg/min/cm², more preferably, between about 0.1 mg/min/cm² and about 5 mg/min/cm², more preferably still, between about 0.1 mg/min/cm² and about 2 mg/min/cm², more preferably still, between about 0.1 mg/min/cm² and about 1.5 mg/min/cm², most preferably, between about 0.1 mg/min/cm² and about 1.25 mg/min/cm².

As defined herein, the dissolution rate of a pharmaceutical agent is determined by the method as described in USP 26, NF21, p.2333.

The low solubility and/or low dissolution rate pharmaceutical agents may be incorporated into the drug composition and/or dosage forms of the present invention in amounts in the range of from about 1 milligram to about 750 milligrams, preferably in the range of from about 5 mg to about 250 mg, more preferably in the range of from about 10 mg to about 250 mg.

An “immediate-release dosage form” refers to a dosage form that releases greater than or equal to about 80% of the pharmaceutical agent in less than or equal to about 1 hour.

By “sustained release” is meant continuous release of a pharmaceutical agent over a prolonged period of time.

By “controlled release” is meant continuous release of a pharmaceutical agent over a prolonged period of time, wherein the pharmaceutical agent is released at a controlled rate over a controlled period of time.

By “prolonged period of time” is meant a continuous period of time of greater than about 1 hour, preferably, greater than about 4 hours, more preferably, greater than about 8 hours, more preferably greater than about 10 hours, more preferably still, greater than about 14 hours, most preferably, greater than about 14 hours and up to about 24 hours.

As used herein, unless otherwise noted, “rate of release” or “release rate” of a drug refers to the quantity of drug released from a dosage form per unit time, e.g., milligrams of drug released per hour (mg/hr). Drug release rates for dosage forms are typically measured as an in vitro rate of drug release, i.e., a quantity of drug released from the dosage form per unit time measured under appropriate conditions and in a suitable fluid.

The release rates referred to herein are determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

As used herein a drug release rate obtained at a specified time refers to the in vitro release rate obtained at the specified time following implementation of the release rate test. The time at which a specified percentage of the drug within a dosage form has been released from said dosage form is referred to as the “Tₓ” value, where “x” is the percent of drug that has been released. For example, a commonly used reference measurement for evaluating drug release from dosage forms is the time at which 70% of drug within the dosage form has been released. This measurement is referred to as the “T₇₀” for the dosage form. Preferably, T₇₀ is greater than or equal to about 8 hours, more preferably, T₇₀ is greater than or equal to about 12 hours, more preferably still, T₇₀ is greater than to equal to about 16 hours, most preferably, T₇₀ is greater than or equal to about 20 hours. In one embodiment, T₇₀ is greater than or equal to about 12 hours and less than about 24 hours. In another embodiment, T₇₀ is greater than or equal to about 8 hours and less than about 16 hours.

By “C” is meant the concentration of drug in blood plasma, or serum, of a subject, generally expressed as mass per unit volume, typically nanograms per milliliter. For convenience, this concentration may be referred to herein as “drug plasma concentration”, “plasma drug concentration” or “plasma concentration” which is intended to be inclusive of a drug concentration measured in any appropriate body fluid or tissue. The plasma drug concentration at any time following drug administration is referenced as Cₜᵢₘₑ, as in C₉ₕ or C₂₄ₕ, etc.

As used herein, unless otherwise noted, the term “zero order rate of release” shall mean a rate of release wherein the amount of drug released as a function of time is substantially constant. More particularly, the rate of release of drug as a function of time shall vary by less than about 30%, preferably, less than about 20%, more preferably, less than about 10%, most preferably, less than about 5%, wherein the measurement is taken over the period of time wherein the cumulative release is between about 25% and about 75%, preferably, between about 25% and about 90%.

As used herein unless otherwise noted, the term “ascending rate of release” shall mean a rate of release wherein the amount of drug released as a function of time increases over a period of time, preferably continuously and gradually. Preferably, the rate of drug released as a function of time increases in a steady (rather than step-wise) manner. More preferably, an ascending rate of release may be characterized as follows. The rate of release as a function of time for a dosage form is measured and plotted as % drug release versus time (cumulative plot) or as milligrams of drug released/hour versus time (release rate plot). An ascending rate of release is characterized by an average rate (expressed in mg of drug per hour) wherein the rate within a given two hour span is higher as compared with the previous two hour time span, over the period of time of about 2 hours to about 12 hours, preferably, about 2 hours to about 18 hours, more preferably about 4 hours to about 12 hours, more preferably still, about 4 hours to about 18 hours. Preferably, the increase in average rate is gradual such that less than about 30% of the dose is delivered during any 2 hour interval, more preferably, less than about 25% of the dose is delivered during any 2 hour interval. Preferably, the ascending release rate is maintained until at least about 50%, more preferably until at least about 75% of the drug in the dosage form has been released.

One skilled in the art will recognize that as the increase in the area under the curve increases (e.g from 1% to 10%), the total time over which the drug is released from the dosage form will necessarily decrease and as such the determination of ascending rate of release will span a shorter overall period of time.

When referring to a dosage form, “high dosage” shall mean a dosage form wherein the pharmaceutical agent, preferably a low solubility and/or low dissolution rate pharmaceutical agent, is present in an amount greater than or equal to about 20%, preferably greater than or equal to about 30%, more preferably greater than or equal to about 50%, by weight of drug compositions within the dosage form.

As used herein, the term “therapeutically effective amount” shall mean that amount of pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “subject” as used herein, refers to an animal, preferably, a mammal, most preferably, a human, who has been the object of treatment, observation or experiment.

As used herein, unless otherwise noted, the term “structural polymer” shall mean any component, for example a polymer or sugar, which is capable of water absorption and which may increase the viscosity of the drug compositions and/or may impart osmotic activity to the drug composition and/or may act as a suspending agent for the drug composition. Suitable examples of structural polymers include, but are not limited to poly(alkyleneoxide polymers of between 100,000 and 750,000 molecular weight, including polyethylene oxide (such as POLYOX® N80; POLYOX® N10, POLYOX N750, and the like); polymethylene oxide, polybutylene oxide and polyhexylene oxide, and poly(carboxymethylcellulose) of 40,000 to 400,000 number average molecular weight, represented by poly(alkali carboxymethylcellulose), poly(sodium carboxymethylcellulose), poly(potassium carboxymethylcellulose), poly(litihium carboxymethylcellulose), and the like. Suitable example also include, but are not limited to sugars such as maltrodextrins (such as MALTRIN M040, MALTRIN M100, MALTRIN M150, MALTRIN M200, MALTRIN M250, and the like); sugars comprising lactose, glucose, raffinose, sucrose, mannitol, sorbitol and the like. Suitable examples also include, but are not limited to polyvinylpyrrolidone (PVP) (such as PVPs of grades 12PF or K2932, and the like); hydroxypropylcellulose; hydroxy propyl alkylcellulose of 9200 to 125,000 average molecular weight represented by hydroxypropyl ethylcellulose, hydroxypropoyl methylcellulose, hydroxypropyl butylcellulose, hydroxypropyl pentylcellulose, and the like; polyvinyl pyrrolisone vinyl acetate co-polymers; and poly(vinylpyrrolidone) of up to 1,000,000 average molecular weight. Preferably, the structural polymer is a polyethylene oxide polymers of between 100,000 and 300,000 molecular weight. More preferably, the structural polymer is POLYOX® N80.

Preferably, the structural polymer is selected from MALTRIN M100, POLYOX N10 and POLYOX N80, more preferably, the structural polymer is POLYOX N80.

As used herein, unless otherwise noted, the term “solubilizing agent” shall mean any component which increases the solubility and/or dissolution rate of a pharmaceutical agent. Preferably, the solubilizing agent is a surfactant. Such surfactants are known in the art.

As used herein, unless otherwise noted, the term “osmopolymer” shall mean a swellable, hydrophilic polymer that interacts with water and swells or expands to a high degree, typically exhibiting a 2-50 fold volume increase. Suitable examples, include but are not limited to poly(alkylene oxide) of 1 million to 15 million number-average molecular weight, as represented by poly(ethylene oxide), poly(alkali carboxymethylcellulose) of 500,000 to 3,500,000 number-average molecular weight, wherein the alkali is sodium, potassium or lithium; polymers that form hydrogels, such as CARBOPOL® acidic carboxypolymer, a polymer of acrylic cross-linked with a polyallyl sucrose, also known as carboxypolymethylene, and carboxyvinyl polymer having a molecular weight of 250,000 to 4,000,000; CYANAMER® polyacrylamides; cross-linked water swellable indenemaleic anhydride polymers; GOOD-RITE® polyacrylic acid having a molecular weight of 80,000 to 200,000; AQUA-KEEPS® acrylate polymer polysaccharides composed of condensed glucose units, such as diester cross-linked polygluran; and the like.

As used herein, unless otherwise noted, the terms “osmoagent” and “osmotically active agent” shall mean an agent which exhibits an osmotic activity gradient across a semi-permeable membrane. Suitable osmoagents include, but are not limited to, sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts, carbohydrates, and the like.

Preferred structural polymer chemical and commercial/tradenames may be used interchangeably throughout the specification herein. For clarity the following is a listing of said structural polymer chemical and corresponding commercial/tradenames.

| Chemical Name | Tradename(s) |
|---|---|
| Polyethylene oxide | POLYOX ® N10 |
| of 100,000 molecular weight | |
| Polyethylene oxide | POLYOX ® N80 |
| of 200,000 molecular weight | |
| Polyethylene oxide | POLYOX ® N 750 |
| of 300,000 molecular weight | |
| Polyethylene oxide | POLYOX ® N 12K |
| of 1,000,000 molecular weight | |
| Polyethylene oxide | POLYOX ® N 60K |
| of 2,000,000 molecular weight | |
| Polyethylene oxide | POLYOX ® 303 |
| of 7,000,000 molecular weight | |

In an embodiment of the present invention, the dosage form is an osmotic dosage form. In another embodiment of the present invention, the dosage form is a controlled release dosage form. Preferably, the dosage form is an osmotic controlled release dosage form, preferably for oral administration.

In an embodiment of the present invention there is provided a dosage form comprising a drug composition, wherein the drug composition comprises (i) a low solubility and/or low dissolution rate free acid pharmaceutical agent, and (ii) a pharmaceutically acceptable salt of the pharmaceutical agent and wherein the low solubility and/or low dissolution rate free acid pharmaceutical agent and/or its pharmaceutically acceptable salt thereof are present in an amount in the range of about 1 milligram to about 750 milligrams, preferably about 5 milligrams to about 250 milligrams, more preferably about 10 milligrams to about 250 milligrams. In another embodiment of the present invention is a dosage form comprising two drug compositions as described herein, wherein the sum of the amount of low solubility and/or low dissolution rate free acid pharmaceutical agent and/or its pharmaceutically acceptable salt present within the drug compositions is in the range of about 1 milligram to about 750 milligrams, preferably about 5 milligrams to about 250 milligrams, more preferably about 10 milligrams to about 250 milligrams.

In another embodiment of the present invention is a dosage form comprising a drug composition, wherein the drug composition comprises topiramate, and a pharmaceutically acceptable salt thereof, and wherein the topiramate and/or its pharmaceutically acceptable salt is present in an amount in the range of about 1 milligram to about 750 milligrams, preferably about 5 milligrams to about 250 milligrams, more preferably about 10 milligrams to about 250 milligrams, more preferably still, topiramate and/or its pharmaceutically acceptable salt is present in an amount selected from 2 mg, 10 mg, 20 mg, 40 mg, 45 mg, 80 mg, 90 mg, 120 mg, 135 mg, 160 mg, 180 mg or 200 mg.

There are many approaches to achieving sustained release or controlled release of drugs from oral dosage forms known in the art. These different approaches may include, but are not limited to, for example, diffusion systems such as reservoir devices and matrix devices, dissolution systems such as encapsulated dissolution systems (including, for example, “tiny time pills”) and matrix dissolution systems, combination diffusion/dissolution systems and ion-exchange resin systems as described in Remington's Pharmaceutical Sciences, 18th ed., pp. 1682-1685, (1990). Pharmaceutical agent dosage forms that operate in accord with these other approaches are encompassed by the scope of the present invention to the extent that said dosage form comprise a pharmaceutical agent and a solubilizing agent and/or produce a substantially zero order rate of release, a substantially ascending rate of release or a rate of release which results in a substantially ascending drug plasma concentration.

Sustained release or controlled release dosage forms may be prepared as osmotic dosage forms. Osmotic dosage forms utilize osmotic pressure to generate a driving force for imbibing fluid into a compartment formed, at least in part, by a semi-permeable wall that permits free diffusion of water but not drug or other components. A significant advantage to osmotic systems is that operation is pH-independent and thus continues at the osmotically determined rate throughout an extended time period, even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated in its entirety by reference herein. In particular, the following U.S. patents, owned by the assignee of the present application, ALZA Corporation, directed to osmotic dosage forms: U.S. Pat. Nos. 3,845,770; 3,916,899; 3,995,631; 4,008,719; 4,111,202; 4,160,020; 4,327,725; 4,519,801; 4,578,075; 4,681,583; 5,019,397; and 5,156,850. Drug delivery devices (i.e. dosage forms) in which a drug composition is delivered as a slurry, suspension or solution from a small exit orifice by the action of an expandable layer are described in U.S. Pat. Nos. 5,633,011; 5,190,765; 5,252,338; 5,620,705; 4,931,285; 5,006,346; 5,024,842; and 5,160,743. Such osmotic dosage forms generally comprise a drug layer, an optional push layer, a semi-permeable membrane which encompasses the drug and push layers and one or more exit orifices.

In the aqueous environment of the gastrointestinal (GI) tract, water is imbibed through the semi-permeable membrane of the osmotic dosage form, at a controlled rate. This causes the push layer to swell and the drug composition(s) to hydrate and form viscous, but deformable, masses. The push layer expands against the drug composition(s), which are pushed out through the orifice. The drug composition(s) exit the system through the exit orifice in the membrane over prolonged periods of time as water from the gastrointestinal tract is imbibed into the delivery system. At the completion of drug release, the biologically inert components of the dosage form are eliminated as a tablet shell.

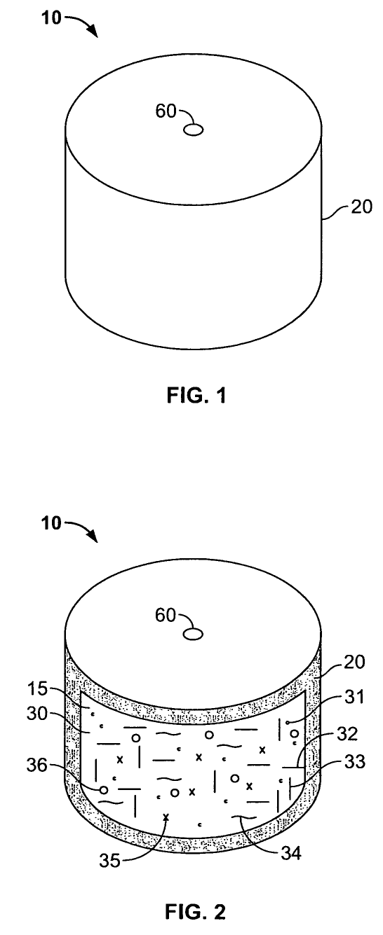

FIG. 1 is a perspective view of one embodiment of a sustained release osmotic dosage form in a standard biconvex round shaped tablet. Dosage form 10 comprises a semi-permeable wall 20 that surrounds and encloses an internal compartment (not seen in FIG. 1). The internal compartment comprises a drug composition comprising a pharmaceutical agent and a solubilizing agent. Semi-permeable wall 20 is provided with at least one exit orifice 60 for connecting the internal compartment with the exterior environment of use. Accordingly, following oral ingestion of dosage form 10, water is imbibed through semi-permeable wall 20 and the pharmaceutical agent/drug composition is released through exit 60.

While the geometrical embodiment in FIG. 1 illustrates a standard biconvex round shaped tablet, the dosage forms of the present invention may embrace other geometries including, a capsule shaped caplet, oval, triangular and other shapes designed for oral administration, including buccal or sublingual dosage forms.

FIG. 2 is a cutaway view of FIG. 1 showing internal compartment 15 containing a single drug composition 30, wherein the drug composition 30 comprises a low solubility and/or low dissolution rate free acid pharmaceutical agent 31 and a pharmaceutically acceptable salt 33 thereof in an admixture with selected excipients. The excipients may be selected to provide an osmotic activity gradient for driving fluid from an external environment through semi-permeable wall 20 for forming a deliverable drug composition upon imbibition of fluid and/or for other performance and/or manufacturing purposes.

In another embodiment of the present invention, as shown in FIG. 2, the drug composition comprises a low solubility and/or low dissolution rate free acid pharmaceutical agent 31, a pharmaceutically acceptable salt 33 and a structural polymer 32 (represented by horizontal dashed lines).

Drug composition 30 excipients may further optionally include a lubricant 34 (represented by horizontal wavy lines), an osmotically active agent, also known as an osmoagent 35 (represented by “X” symbols) and/or a suitable binder 36 (represented by large circles).

In operation, following oral ingestion of dosage form 10, the osmotic activity gradient across the semi-permeable wall 20 causes water of the gastrointestinal tract to be imbibed through the semi-permeable wall 20, thereby forming a deliverable drug composition, e.g., a solution or suspension or hydrogel, within the internal compartment. The deliverable drug composition is then released through the exit orifice 60 as water continues to enter the internal compartment. As release of the drug composition occurs, water continues to be imbibed thereby driving continued release. In this manner, drug is released in a sustained and continuous manner over an extended time period.

In an embodiment of the present invention is a osmotic controlled release dosage form comprising

(a) a core comprising a first drug composition, wherein the first drug composition comprises a low solubility and/or low dissolution rate free acid pharmaceutical agent and/or its pharmaceutically acceptable salt;

(b) a semi-permeable wall surrounding the core; and

(c) an exit orifice through the semi-permeable wall for releasing the first drug composition from the dosage form over a prolonged period of time.

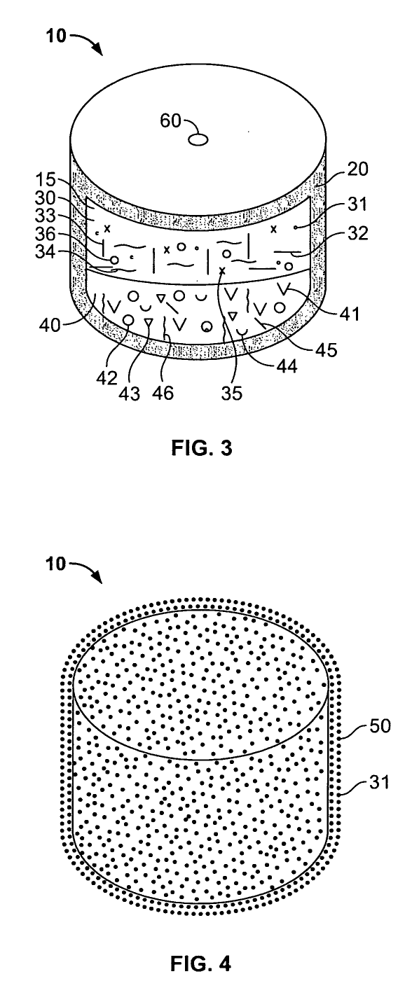

FIG. 3 is a cutaway view of FIG. 1 with an alternate embodiment of internal compartment 15, wherein the internal compartment comprises a bi-layer configuration. In this embodiment, internal compartment 15 contains a bi-layered compressed core having a first drug composition 30 and a push layer 40. Drug composition 30, as described above with reference to FIG. 1 and 2, comprises a low solubility and/or low dissolution rate free acid pharmaceutical agent 31 and a pharmaceutically acceptable salt 33, in an admixture with further, optional excipients.

As is described in more detail below, the second component, push layer 40, comprises osmotically active component(s), but does not contain any pharmaceutical agent. In an embodiment of the present invention, push layer 40 comprises osmopolymer 41. Preferably, the components in push layer 40 comprise an osmoagent 42 (represented by very large circles) and one or more osmopolymers 41 (represented by “V” symbols).

Additionally, optional excipients within push layer 40, may include binder 43 (represented by down-ward triangles), lubricant 44 (represented by upward semi-circles), antioxidant 45 (represented by diagonal lines) and/or colorant 46 (represented by vertical wavy lines).

As water is imbibed through the semi-permeable wall 20, the osmopolymer(s) within push layer 40 swell and push against drug composition 30 to thereby facilitate release of the drug composition through the exit orifice 60 and thus the pharmaceutical agent from the dosage form.

In an embodiment of the present invention, drug composition 30, as described with reference to FIGS. 2 and 3 comprises a low solubility and/or low dissolution rate free acid pharmaceutical agent 31 and a pharmaceutically acceptable salt 33 in an admixture with further, optional, selected excipients. The excipients may be one or more selected from a structural polymer 32, lubricant 34, an osmoagent 35 and/or a binder 36.

In another embodiment of the present invention, push layer 40, as described with reference to FIG. 3, comprises osmotically active components, more specifically an osmoagent 42 and an osmopolymer 41, but does not contain any pharmaceutical agent.

FIG. 4 is a view of another embodiment of the present invention, a biconvex round standard tablet as in FIG. 1, wherein the tablet includes a further, optional immediate release coating 50 of a pharmaceutical agent, preferably topiramate, covering the dosage form of FIG. 1, 2 or 3.

More specifically, dosage form 10 of FIG. 4 comprises an overcoat 50 on the outer surface of semi-permeable wall 20 of dosage form 10. Overcoat 50 is a drug composition comprising about 10 μg to about 500 mg of low solubility and/or low dissolution rate free acid pharmaceutical agent 31, preferably, overcoat 50 comprises about 10 μg to about 200 mg of low solubility and/or low dissolution rate free acid pharmaceutical agent 31, more preferably, overcoat 50 comprises about 5 mg to about 100 mg of low solubility and/or low dissolution rate free acid pharmaceutical agent 31 and from about 5 mg to about 200 mg of a pharmaceutically acceptable carrier selected from the group consisting of alkylcellulose, hydroxyalkylcellulose and hydroxypropylalkylcellulose. The overcoat of pharmaceutically acceptable carrier preferably may comprise a polymer or copolymer such as methylcellulose, hydroxyethylcellulose, hydroxybutylcellulose, hydroxypropylcellulose, hydroxypropylmethylcellulose, hydroxypropylethylcellulose and hydroxypropylbutylcellulose, polyvinyl pyrrolidone/vinyl acetate copolymer, polyvinyl alcohol-polyethylene graft copolymer, and the like. Overcoat 50 provides immediate release of the low solubility and/or low dissolution rate free acid pharmaceutical agent 31, as overcoat 50 dissolves in the presence of gastrointestinal fluid and concurrently therewith delivers low solubility and/or low dissolution rate free acid pharmaceutical agent 31 into the gastrointestinal tract for immediate therapy. Low solubility and/or low dissolution rate free acid pharmaceutical agent 31 in overcoat 50 can be the same or different than low solubility and/or low dissolution rate free acid pharmaceutical agent 31 in drug composition 30. Preferably low solubility and/or low dissolution rate free acid pharmaceutical agent 31 in overcoat 50 is the same as low solubility and/or low dissolution rate free acid pharmaceutical agent 31 in drug composition 30.

In an embodiment of the invention, dosage forms are provided that result in an ascending rate of release of the low solubility and/or low dissolution rate free acid pharmaceutical agent taken together with its pharmaceutically acceptable salt. This may be accomplished by controlling levels of the free acid and its salt in different drug layers of the system. Such an ascending rate of release could not be easily accomplished using only the free acid or only the salt in both layers, because of the solubility or dissolution rate problems with the free acid noted above, and because the higher solubility of the salt would provide very rapid mixing between the drug layers with a resulting zero-order release profile.

In another embodiment of the present invention is a dosage form comprising two drug compositions, wherein each drug composition comprises a low solubility and/or low dissolution rate free acid pharmaceutical agent and/or a pharmaceutically acceptable salt thereof, and wherein the sum of the amount of pharmaceutical agent and pharmaceutically acceptable salt thereof within the drug compositions is in the range of about 1 milligram to about 750 milligrams, preferably about 5 milligrams to about 250 milligrams, more preferably about 10 milligrams to about 250 milligrams, more preferably still, the pharmaceutical agent is present in an amount selected from 10 mg, 20 mg, 40 mg, 45 mg, 80 mg, 90 mg, 120 mg, 135 mg, 160 mg, 180 mg or 200 mg.

In another embodiment of the present invention is a osmotic controlled release dosage form comprising

(a) a core comprising a first drug composition, a second drug composition and a push layer, wherein the first and second drug composition each comprise a low solubility and/or low dissolution rate free acid pharmaceutical agent and/or its pharmaceutically acceptable salt;

(b) a semi-permeable wall surrounding the core; and

(c) an exit orifice through the semi-permeable wall for releasing the first and second drug compositions from the dosage form over a prolonged period of time.

In an embodiment of the present invention, the total amount and/or concentration of the low solubility and/or low dissolution rate free acid pharmaceutical agent and its pharmaceutically acceptable salt within the first drug composition is less than the total amount and/or concentration of the low solubility and/or low dissolution rate free acid pharmaceutical agent and its pharmaceutically acceptable salt within the second drug composition.

In an embodiment of the present invention, the low solubility and/or low dissolution rate free acid pharmaceutical agents in the first and second drug compositions are independently selected. Preferably, the low solubility and/or low dissolution rate free acid pharmaceutical agents in the first and second drug compositions are the same.

In an embodiment of the present invention, the push layer comprises an osmopolymer. In another embodiment of the present invention, the push layer comprises and osmopolymer and an osmoagent.

In an embodiment of the present invention, the osmotic controlled release dosage form releases the low solubility and/or low dissolution rate free acid pharmaceutical agent and/or its pharmaceutically acceptable salt over a prolonged period of time, preferably over greater than 4 hours, more preferably, over greater than about 8 hours, more preferably still, over greater than about 10 hours, most preferably, over greater than about 14 hours. In another embodiment of the present invention, the osmotic controlled release dosage form releases the low solubility and/or low dissolution rate free acid pharmaceutical agent and/or its pharmaceutically acceptable salt over a prolonged period of time greater than about 14 hours and up to about 24 hours.

In an embodiment of the present invention, the osmotic controlled release dosage form releases low solubility and/or low dissolution rate free acid pharmaceutical agent and/or its pharmaceutically acceptable salt at a substantially ascending rate of release. In yet another embodiment of the present invention, the osmotic controlled release dosage form releases low solubility and/or low dissolution rate free acid pharmaceutical agent and/or its pharmaceutically acceptable salt at a rate that results in a substantially ascending drug plasma concentration.

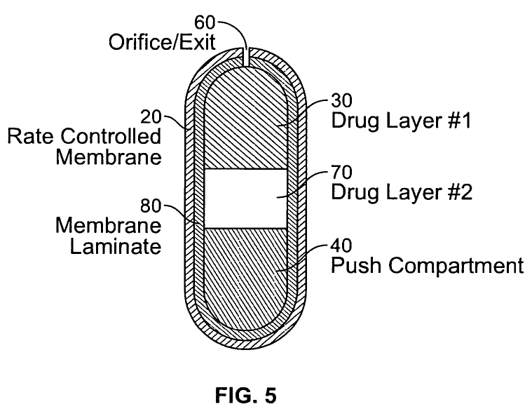

FIG. 5 shows an embodiment of the present invention, illustrating an open view of a tri-layer capsule shaped osmotic dosage form. FIG. 5 illustrates a capsule shaped tablet embodiment of the present invention comprising a first drug composition 30, a second drug composition 70 and a push layer 40. The capsule shaped core (comprising the first and second drug compositions and the push layer) is enveloped by semi-permeable membrane 20. The dosage form further comprises at least one exit orifice 60, which exposes the first drug composition 30 to the environment of use. The dosage form in FIG. 5 further comprises an additional, optional inner membrane 80 that may function as a flow-promoting layer and/or as a smoothing layer and/or contribute to the control of the rate of imbibition of water into the dosage form.

In an embodiment of the present invention, as described in FIG. 5, the amount and/or concentration of the drug in the first drug composition 30 is different than the amount and/or concentration of low solubility and/or low dissolution rate free acid pharmaceutical agent and/or its pharmaceutically acceptable salt in second drug composition 70. In another embodiment of the present invention, the amount and/or concentration of drug in the first drug composition 30 is less than the amount and/or concentration of low solubility and/or low dissolution rate free acid pharmaceutical agent and/or its pharmaceutically acceptable salt in second drug composition 70. Preferably, the amount and/or concentration of drug in the first drug composition 30 is less than the amount and/or concentration of drug in the second drug composition 70. More preferably, the amounts and/or concentrations of low solubility and/or low dissolution rate free acid pharmaceutical agent and/or its pharmaceutically acceptable salt in the first and second drug compositions are selected to yield a substantially ascending rate of release of the pharmaceutical agent.

The dosage form illustrated in FIG. 5 may further comprise additional drug compositions having varying low solubility and/or low dissolution rate free acid pharmaceutical agent and/or its pharmaceutically acceptable salt amounts and/or concentrations, to provide alternate release rates and/or patterns and/or to achieve alternate drug plasma concentration profiles that may be preferred.

In a preferred embodiment of the present invention, the low solubility and/or low dissolution rate free acid pharmaceutical agent and/or its pharmaceutically acceptable salt in drug layer 30 is present in a therapeutically effective amount. In another embodiment of the present invention, the total amount of low solubility and/or low dissolution rate free acid pharmaceutical agent and/or its pharmaceutically acceptable salt present in the drug composition or compositions of the dosage forms of the present invention, is equal to or greater than the therapeutically effective, recommended or desired daily dosage.

In an embodiment of the present invention, the low solubility and/or low dissolution rate free acid pharmaceutical agent and/or its pharmaceutically acceptable salt in drug composition 30 (or wherein the dosage form comprises more than one drug composition, the low solubility and/or low dissolution rate free acid pharmaceutical agent and/or its pharmaceutically acceptable salt in the combined drug compositions) is present in an amount equal to or greater than the recommended or desired daily dosage of the low solubility and/or low dissolution rate free acid pharmaceutical agent and/or its pharmaceutically acceptable salt to be administered to a patient in need thereof, thereby permitting once-a-day or less frequent dosing.

Wherein the dosage form contains more than one drug composition, as for example in FIG. 5 wherein two drug compositions 30 and 70 are present, each drug composition comprises independently selected (a) low solubility and/or low dissolution rate free acid pharmaceutical agent 31, and (b) its pharmaceutically acceptable salt 33. Each drug composition may further optionally contain independently selected structural polymer 32 and/or one or more independently selected excipients as hereinafter described.

Wherein two or more drug compositions are present within the dosage forms of the present invention, the daily dosage of the low solubility and/or low dissolution rate free acid pharmaceutical agent and/or its pharmaceutically acceptable salt is present in divided amounts. For example, if the total dosage of the pharmaceutical agent (free acid plus salt) is 400 mg, and the dosage form comprises two drug compositions (e.g. drug compositions 30 and 70 as exemplified in FIG. 5), then the sum of the amount of low solubility and/or low dissolution rate free acid pharmaceutical agent and/or its pharmaceutically acceptable salt in the first drug composition plus the amount of low solubility and/or low dissolution rate free acid pharmaceutical agent and/or its pharmaceutically acceptable salt in the second drug composition will total 400 mg or more.

Preferably, any low solubility and/or low dissolution rate free acid pharmaceutical agent according to the invention is present in the drug composition in micronized form. Preferably, the micronized low solubility and/or low dissolution rate free acid pharmaceutical agent has a nominal particle size of less than about 200 microns, more preferably less than about 100 microns, most preferably, less than about 50 microns.

In an embodiment, to achieve a substantially zero order release rate profile, the weight ratio of low solubility and/or low dissolution rate free acid pharmaceutical agent to its pharmaceutically acceptable salt in drug composition 30 is preferably, in the range of from about 0.25 to about 2.0, more preferably, in the range of from about 0.3 to about 1.5, more preferably still, in the range of from about 0.5 to about 1.0. These ranges are preferably applied to bi-layer osmotic controlled release dosage forms.

In one embodiment, to achieve a substantially ascending release rate profile, the first drug composition 30 (as shown, for instance, in FIG. 5) comprises low solubility and/or low dissolution rate free acid pharmaceutical agent and is substantially free from its pharmaceutically acceptable salt, and the second drug composition 70 (as shown, for instance, in FIG. 5) comprises the pharmaceutically acceptable salt and is substantially free from the low solubility and/or low dissolution rate free acid pharmaceutical agent.

In another embodiment, to achieve a substantially ascending release rate profile, the first drug composition 30 (as shown, for instance, in FIG. 5) comprises low solubility and/or low dissolution rate free acid pharmaceutical agent and its pharmaceutically acceptable salt in a weight ratio of about 0.5 to about 5.0 acid:salt, preferably about 1.0 to about 4.0 acid:salt, more preferably about 2.0 to 3.0 acid:salt, and the second drug composition 70 (as shown, for instance, in FIG. 5) comprises the low solubility and/or low dissolution rate free acid pharmaceutical agent and its pharmaceutically acceptable salt in a weight ratio of about 0.15 to about 2.0 acid:salt, preferably about 0.3 to about 1.5 acid:salt, more preferably about 0.5 to 1.0 acid:salt. These ranges are preferably applied to bi-layer osmotic controlled release dosage forms.

Structural polymer 32 (as shown in FIGS. 2 and 3) comprises any component, for example a hydrophilic polymer, which provides cohesiveness to the blend so durable tablets can be made. The structural polymer may also form a hydrogel for viscosity control during the operation of the delivery system. The structural polymer further suspends the drug particles to promote partial or complete solubilization of the drug within the dosage form prior to delivery from the dosage form.

The molecular weight of the structural polymer 32 may be chosen to impart desired properties to the dosage form, and more particularly to the drug compositions within the dosage form. High molecular weight polymers are used to produce a slow hydration rate and slow delivery of drug, whereas low molecular weight polymers produce a faster hydration rate and faster release of drug. A blend of high and low molecular weight structural polymers produces an intermediate delivery rate.

Structural polymer 32 is a hydrophilic polymer particle in the drug composition that contributes to the controlled delivery of active agent. Representative examples of suitable structural polymers include, but are not limited to, poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 1,000,000 400,000 number-average molecular weight, represented by poly(alkali carboxymethylcellulose), poly(sodium carboxymethylcellulose), poly(potassium carboxymethylcellulose) poly(calcium carboxymethylcellulose), and poly(lithium carboxymethylcellulose). The drug composition may alternatively comprise a hydroxypropylalkylcellulose of 9,200 to 125,000 number-average molecular weight for enhancing the delivery properties of the dosage form such as hydroxypropylethylcellulose, hydroxypropylmethylcellulose, hydroxypropylbutylcellulose, hydroxypropylpentylcellulose, and the like; and/or a poly(vinylpyrrolidone) of 7,000 to 75,000 number-average molecular weight for enhancing the flow properties of the dosage form. Preferred structural polymers are the poly(ethylene oxide) polymers of 100,000-300,000 number average molecular weight. Structural polymers that erode in the gastric environment, i.e., bioerodible structural polymers, are especially preferred.

Other structural polymers that may be incorporated into drug composition 30 include carbohydrates that exhibit sufficient osmotic activity to be used alone or with other osmoagents. Such carbohydrates comprise monosaccharides, disaccharides and polysaccharides. Representative examples include, but are not limited to, maltodextrins (i.e., glucose polymers produced by the hydrolysis of grain starch such as rice or corn starch) and the sugars comprising lactose, glucose, raffinose, sucrose, mannitol, sorbitol, zylitol and the like. Preferred maltodextrins are those having a dextrose equivalence (DE) of about 20 or less, preferably maltodextrins with a DE ranging from about 4 to about 20, and more preferably from about 9 to about 20. Maltodextrins having a DE of about 9-12 and molecular weight of about 1,600 to 2,500 are preferred.

The carbohydrates described above, preferably the maltodextrins, may be used in the drug composition 30 without the addition of an osmoagent, to yield the desired release of low solubility and/or low dissolution rate free acid pharmaceutical agent and/or its pharmaceutically acceptable salt from the dosage form, while providing a therapeutic effect over a prolonged period of time and up to 24 hours with once-a-day dosing.

Preferably, the structural polymer is selected form the group consisting of poly(ethylene oxide), poly(methylene oxide), poly(butylene oxide) and poly(hexylene oxide); poly(carboxymethylcellulose), poly(alkali carboxymethylcellulose), poly(sodium carboxymethylcellulose), poly(potassium carboxymethylcellulose) poly(calcium carboxymethylcellulose), poly(lithium carboxymethylcellulose), hydroxypropylcellulose, hydroxypropylethylcellulose, hydroxypropylmethylcellulose, hydroxypropylbutylcellulose, hydroxypropylpentylcellulose, poly(vinylpyrrolidone), a bioerodible structural polymer, maltodextrin, polyvinyl pyrrolidone, a polyvinylpyrrolidone vinyl acetate copolymer, lactose, glucose, raffinose, sucrose, mannitol, sorbitol, zylitol and mixtures thereof.

More preferably, the structural polymer is selected from the group consisting of MALTRIN M100, POLYOX N10 and POLYOX N80, most preferably, the structural polymer is POLYOX N80.

It has been further found that, when present, the structural polymer and pharmaceutically acceptable salt are preferably present in the drug composition in a certain amounts. Preferably, the structural polymer should be present in an amount less than or equal to about 90% by weight of the drug composition and the pharmaceutically acceptable salt should be present in amount between 0 and about 50% by weight of the drug composition. Preferably, for high dosages, the structural polymer should be present in an amount less than or equal to about 30% by weight of the drug composition, more preferably in an amount less than about 20% by weight of the drug composition; and the pharmaceutically acceptable salt should be present in amount greater than or equal to about 15% by weight of the drug composition, more preferably, in an amount greater than or equal to about 25% by weight of the drug composition, more preferably still, in an amount greater than or equal to about 35% by weight of the drug composition, most preferably, in an amount greater than or equal to about 40% by weight of the drug composition.

Lubricant 34 may optionally be included in the drug composition as represented by a horizontal wavy line in FIG. 2 and FIG. 3. Lubricant 34 is used during tablet manufacture to prevent adherence to die walls or punch faces. Typical lubricants include, but are not limited to, magnesium stearate, sodium stearate, stearic acid, calcium stearate, magnesium oleate, oleic acid, potassium oleate, caprylic acid, sodium stearyl fumarate, and magnesium palmitate or blends of such lubricants. The amount of lubricant present in the drug composition is preferably, in the range of from about 0.01 to about 20 mg.

Binder 36, preferably a therapeutically acceptable vinyl polymer binder, may also be optionally included in the drug composition as represented by small circles in FIG. 2 and FIG. 3. Representative binders include, but are not limited to vinyl polymer binder, acacia, starch and gelatin. Wherein the binder is a vinyl polymer, the vinyl polymer comprises a 5,000 to 350,000 average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Representative other binders suitable for formulation in the drug composition include, but are not limited to acacia, starch and gelatin. The binder present within the drug composition is preferably, in an amount in the range of from about 0.01 to about 25 mg.

Disintegrants may also be optionally included in the drug composition. Disintegrants may be selected from starches, clays, celluloses, algins and gums and crosslinked starches, celluloses and polymers. Representative disintegrants include, but are not limited to, corn starch, potato starch, croscarmelose, crospovidone, sodium starch glycolate, VEEGUM HV, methylcellulose, agar, bentonite, carboxymethylcellulose, alginic acid, guar gum, low-substituted hydroxypropyl cellulose, microcrystalline cellulose, and the like.

One skilled in the art will recognize that the amounts of low solubility and/or low dissolution rate free acid pharmaceutical agent and/or its pharmaceutically acceptable salt and structural polymer are selected to optimize the characteristics of the drug layer composition. The amounts are selected such that the dosage form maintains structural integrity before administration and upon administration, the drug layer composition hydrates and is capable of being pushed out of the dosage form providing a desired release pattern.

In an embodiment of the present invention is a drug composition, wherein the low solubility and/or low dissolution rate free acid pharmaceutical agent and/or its pharmaceutically acceptable salt is present in amount in the range of about 10 mg to about 200 mg. In further embodiments of the present invention are drug compositions wherein the low solubility and/or low dissolution rate free acid pharmaceutical agent and/or its pharmaceutically acceptable salt is present in 2 mg, 10 mg, 20 mg, 40 mg, 45 mg, 80 mg, 90 mg, 120 mg, 135 mg, 160 mg, 180 mg and 200 mg amount.

In an embodiment of the present invention is a dosage form comprising one or more drug compositions, preferably one to two drug compositions, wherein the total amount of low solubility and/or low dissolution rate free acid pharmaceutical agent and/or its pharmaceutically acceptable salt present within the dosage form (i.e. the total amount present within the drug compositions) is in an amount in the range of about 10 mg to about 200 mg. In further embodiments of the present invention are dosage forms comprising one or two drug compositions wherein the total amount of low solubility and/or low dissolution rate free acid pharmaceutical agent and/or its pharmaceutically acceptable salt present is 10 mg, 20 mg, 40 mg, 45 mg, 80 mg, 90 mg, 120 mg, 135 mg, 160 mg, 180 mg or 200 mg amount.

One skilled in the art will recognize will that wherein the dosage forms of the present invention comprise a first drug composition comprising a low solubility and/or low dissolution rate free acid pharmaceutical agent and/or its pharmaceutically acceptable salt; and a second drug composition comprising a low solubility and/or low dissolution rate free acid pharmaceutical agent and/or its pharmaceutically acceptable salt; then the low solubility and/or low dissolution rate free acid pharmaceutical agent and/or its pharmaceutically acceptable salt in the first and second drug compositions may be the same or different. One skilled in the art will further recognize that additional, optional components within the first and second drug compositions, for example structural polymer, binder, lubricant, and the like, when present in both the first and second drug compositions may similarly be the same or different.

The formulations and processes for the manufacture of the push layer 40, the semi-permeable wall 20 and the exit orifice(s) 60 are well known in the art. The components and processes for the manufacture of the push layer, semi-permeable wall and exit orifice(s) is also briefly described below.

Push layer 40 comprises a displacement composition in contacting, layered arrangement with drug composition 30 as illustrated in FIG. 3. Wherein more than one drug composition is present in the dosage form (as in FIG. 5), the push layer 40 is preferably in contacting, layered arrangement with only one of the drug compositions.

In an embodiment of the present invention push layer 40 comprises and osmopolymer. In another embodiment of the present invention, push layer 40 comprises an osmopolymer and an osmoagent.

Push layer 40 comprises osmopolymer 41 that imbibes water and swells to push the drug composition of the drug layer(s) through the exit orifice of the dosage form. The osmopolymers are swellable, hydrophilic polymers that interact with water and swell or expand to a high degree, typically exhibiting a 2-50 fold volume increase. The osmopolymer can be non-crosslinked or crosslinked. Preferably, push layer 40 comprises from about 20 to about 375 mg of osmopolymer 41, represented by “V” symbols in FIG. 3.

Wherein osmopolymers are present in both the drug composition and the push layer, the osmopolymer 41 in the push layer 40 possesses a higher molecular weight than the osmopolymer in drug composition. For example, such a situation may be found wherein the structural polymer in the drug composition is an osmopolymer.

Representatives of osmopolymers (i.e. fluid-imbibing displacement polymers) comprise members selected from poly(alkylene oxide) of 1 million to 15 million number-average molecular weight, as represented by poly(ethylene oxide), and poly(alkali carboxymethylcellulose) of 500,000 to 3,500,000 number-average molecular weight, wherein the alkali is sodium, potassium or lithium. Examples of alternate osmopolymers comprise polymers that form hydrogels, such as CARBOPOL® acidic carboxypolymer, a polymer of acrylic cross-linked with a polyallyl sucrose, also known as carboxypolymethylene, and carboxyvinyl polymer having a molecular weight of 250,000 to 4,000,000; CYANAMER® polyacrylamides; cross-linked water swellable indenemaleic anhydride polymers; GOOD-RITE® polyacrylic acid having a molecular weight of 80,000 to 200,000; AQUA-KEEPS® acrylate polymer polysaccharides composed of condensed glucose units, such as diester cross-linked polygluran; and the like. Representative polymers that form hydrogels are known to the prior art in U.S. Pat. No. 3,865,108, issued to Hartop; U.S. Pat. No. 4,002,173, issued to Manning; U.S. Pat. No. 4,207,893, issued to Michaels; and in Handbook of Common Polymers, Scott and Roff, Chemical Rubber Co., Cleveland, Ohio.

Push layer 40 further, optionally, comprises an osmotically effective compound, osmoagent 42, represented by large circles in FIG. 3. Preferably, the osmoagent 42 comprises up to about 40% by weight of the push layer, more preferably, from about 5% to about 30% by weight of the push layer, more preferably still, from about 10% to about 30% by weight of the push layer. Osmotically effective compounds are known also as osmoagents and/or as osmotically effective solutes. Preferably, push layer 40 comprises an osmoagent.

Osmoagents 42, which may be found in the drug composition and/or the push layer in the dosage forms of the present invention are those that exhibit an osmotic activity gradient across the wall 20. Suitable osmoagents include, but are not limited to, sodium chloride, potassium chloride, lithium chloride, magnesium sulfate, magnesium chloride, potassium sulfate, sodium sulfate, lithium sulfate, potassium acid phosphate, mannitol, urea, inositol, magnesium succinate, tartaric acid, raffinose, sucrose, glucose, lactose, sorbitol, inorganic salts, organic salts, carbohydrates, and the like.

Push layer 40 may further optionally comprises a pharmaceutically acceptable binder 43, such as a vinyl polymer, represented by triangles in FIG. 3. The vinyl polymer comprises a 5,000 to 350,00 viscosity-average molecular weight, represented by a member selected from the group consisting of poly-n-vinylamide, poly-n-vinylacetamide, poly(vinyl pyrrolidone), also known as poly-n-vinylpyrrolidone, poly-n-vinylcaprolactone, poly-n-vinyl-5-methyl-2-pyrrolidone, and poly-n-vinylpyrrolidone copolymers with a member selected from the group consisting of vinyl acetate, vinyl alcohol, vinyl chloride, vinyl fluoride, vinyl butyrate, vinyl laureate, and vinyl stearate. Push layer 40 preferably contains from about 0.01 to about 25 mg of vinyl polymer.

Push layer 40 may further optionally comprise from 0 to about 5 mg of a nontoxic colorant or dye 46, identified by vertical wavy lines in FIG. 3. Suitable examples of colorant or dye 46 include Food and Drug Administration Colorants (FD&C), such as FD&C No. 1 blue dye, FD&C No. 4 red dye, red ferric oxide, yellow ferric oxide, titanium dioxide, carbon black, indigo, and the like.

Push layer 40 may further optionally comprise lubricant 44, identified by half circles in FIG. 3. Suitable examples include, but are not limited to, a member selected from the group consisting of sodium stearate, potassium stearate, magnesium stearate, stearic acid, calcium stearate, sodium oleate, calcium palmitate, sodium laurate, sodium ricinoleate and potassium linoleate, and blends of such lubricants. The amount of lubricant included in the push layer 40 is preferably in the range of from about 0.01 to about 10 mg.

Push layer 40 may further optionally comprise an antioxidant 45, represented by slanted dashes in FIG. 3, wherein the antioxidant is present to inhibit the oxidation of ingredients within the push layer. Push layer 40 comprises from 0.0 to about 5 mg of an antioxidant. Representative antioxidants include, but are not limited to, ascorbic acid, ascorbyl palmitate, butylated hydroxyanisole, a mixture of 2 and 3 tertiary-butyl-4-hydroxyanisole, butylated hydroxytoluene, sodium isoascorbate, dihydroguaretic acid, potassium sorbate, sodium bisulfate, sodium metabisulfate, sorbic acid, potassium ascorbate, vitamin E, 4-chloro-2,6-ditertiary butylphenol, alpha-tocopherol, and propylgallate.

Semi-permeable wall 20, sometimes also referred to as a membrane, is formed to be permeable to the passage of external water. Semi-permeable wall 20 is also substantially impermeable to the passage of the components of the drug composition and push layer, such as drug, solubilizing agent, structural polymer, osmagent, osmopolymer and the like. As such, wall 20 is semi-permeable. The selectively semi-permeable compositions used for forming the semi-permeable wall 20 are essentially non-erodible and are substantially insoluble in biological fluids during the life of the dosage form.

Representative polymers suitable for forming semi-permeable wall 20 comprise semi-permeable homopolymers, semi-permeable copolymers, and the like. Such materials include, but are not limited to, cellulose esters, cellulose ethers and cellulose ester-ethers. The cellulosic polymers have a degree of substitution (DS) of their anhydroglucose unit of from greater than 0 up to 3, inclusive. Degree of substitution (DS) means the average number of hydroxyl groups originally present on the anhydroglucose unit that are replaced by a substituting group or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkysulfamate, semi-permeable polymer forming groups, and the like, wherein the organic moieties contain from one to twelve carbon atoms, and preferably from one to eight carbon atoms.

Semi-permeable wall 20 may further compromise a semi-permeable polymer selected from the group consisting of cellulose acylate, cellulose diacylate, cellulose triacylate, cellulose acetate, cellulose diacetate, cellulose triacetate, mono-, di- and tri-cellulose alkanylates, mono-, di-, and tri-alkenylates, mono-, di-, and tri-aroylates, and the like. Exemplary polymers include cellulose acetate having a DS in the range of about 1.8 to about 2.3 and an acetyl content in the range of about 32 to about 39.9%; cellulose diacetate having a DS in the range of about 1 to about 2 and an acetyl content in the range of about 21 to about 35%; cellulose triacetate having a DS in the range of about 2 to about 3 and an acetyl content in the range of about 34 to about 44.8%; and the like. Preferred cellulosic polymers include cellulose propionate having a DS of about 1.8 and a propionyl content of about 38.5%; cellulose acetate propionate having an acetyl content in the range of about 1.5 to about 7% and an acetyl content in the range of about 39% to about 42%; cellulose acetate propionate having an acetyl content in the range of about 2.5% to about 3%, an average propionyl content in the range of about 39.2% to about 45%, and a hydroxyl content in the range of about 2.8% to about 5.4%; cellulose acetate butyrate having a DS of about 1.8, an acetyl content in the range of about 13% to about 15%, and a butyryl content in the range of about 34% to about 39%; cellulose acetate butyrate having an acetyl content in the range of about 2% to about 29%, a butyryl content in the range of about 17% to about 53%, and a hydroxyl content in the range of about 0.5% to about 4.7%; cellulose triacylates having a-DS in the range of about 2.6 to about 3, such as cellulose trivalerate, cellulose trilamate, cellulose tripalmitate, cellulose trioctanoate and cellulose tripropionate; cellulose diesters having a DS in the range of about 2.2 to about 2.6, such as cellulose disuccinate, cellulose dipalmitate, cellulose dioctanoate, cellulose dicaprylate, and the like; and mixed cellulose esters, such as cellulose acetate valerate, cellulose acetate succinate, cellulose propionate succinate, cellulose acetate octanoate, cellulose valerate palmitate, cellulose acetate heptanoate, and the like. Semi-permeable polymers are known in U.S. Pat. No. 4,077,407, and they can be synthesized by procedures described in Encyclopedia of Polymer Science and Technology, Vol. 3, pp. 325-354 (1964), Interscience Publishers Inc., New York, N.Y.

Additional semi-permeable polymers that may be used for forming semi-permeable wall 20 comprise cellulose acetaldehyde dimethyl acetate; cellulose acetate ethylcarbamate; cellulose acetate methyl carbamate; cellulose dimethylaminoacetate; semi-permeable polyamide; semi-permeable polyurethanes; semi-permeable sulfonated polystyrenes; cross-linked selectively semi-permeable polymers formed by the coprecipitation of an anion and a cation, as disclosed in U.S. Pat. Nos. 3,173,876; 3,276,586; 3,541,005; 3,541,006 and 3,546,142; semi-permeable polymers, as disclosed by Loeb, et al. in U.S. Pat. No. 3,133,132; semi-permeable polystyrene derivatives; semi-permeable poly(sodium styrenesulfonate); semi-permeable poly(vinylbenzyltrimethylammonium chloride); and semi-permeable polymers exhibiting a fluid permeability of 10⁻⁵ to 10⁻² (cc. mil/cm hr.atm), expressed as per atmosphere of hydrostatic or osmotic pressure differences across a semi-permeable wall. The polymers are known to the art in U.S. Pat. Nos. 3,845,770; 3,916,899 and 4,160,020; and in Handbook of Common Polymers, Scott and Roff (1971) CRC Press, Cleveland, Ohio. Wall 20 can optionally be formed as two or more lamina such as described in U.S. Pat. No. 6,210,712.

Preferably, the semi-permeable wall 20 comprises a polymer selected from the group consisting of cellulose acetate and cellulose acetate butyrate.

Semi-permeable wall 20 may further, optionally, comprise a flux-regulating agent. The flux regulating agent is a compound added to assist in regulating the water permeability or flux through semi-permeable wall 20. The flux-regulating agent can be a flux-enhancing agent or a flux-decreasing agent. The flux-regulating agent can therefore be pre-selected to increase or decrease the flux of the external water through the semi-permeable membrane. Flux-regulating agents that produce a marked increase in permeability to fluid such as water are often essentially hydrophilic, while those that produce a marked decrease to fluids such as water are essentially hydrophobic. The amount of flux-regulator in semi-permeable wall 20 when incorporated therein is preferably in the range of from about 0.01% to about 25% by weight or more.

Suitable flux-regulating agents include, but are not limited to, polyhydric alcohols, polyalkylene glycols, polyalkylenediols, polyesters of alkylene glycols, and the like.

Flux enhancers include, but are not limited to, polyethylene glycol 300, 400, 600, 1500, 4000, 6000 and the like; low molecular weight glycols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glycol dipropionate, glycerol acetate esters, and the like. Preferred flux enhancers include the group of difunctional block-copolymer of ethylene oxide and propylene oxide conforming to the general formula OH(C₂H₄O)ₐ(C₃H₆O)_{b}(C₂H₄O)H, known as PLURONIC® co-polymers (sold in pharmaceutical grade under the trade name LUTROL).

Flux-decreasing agents include, but are not limited to, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; polyvinyl acetates, triethyl citrate, Eudragit; insoluble salts such as calcium sulfate, barium sulfate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based wall forming materials, and the like.

Other materials may be further, optionally, included in the semi-permeable wall composition for imparting flexibility and/or elongation properties, i.e. to make semi-permeable wall 20 less brittle and/or to render tear strength to semi-permeable wall 20. Suitable materials include, but are not limited to, phthalate plasticizers such as dibenzyl phthalate, dihexyl phthalate, butyl octyl phthalate, straight chain phthalates of six to eleven carbons, di-isononyl phthalte, di-isodecyl phthalate, and the like. Plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in semi-permeable wall 20 when incorporated therein is preferably in the range of from about 0.01% to about 20% weight, or higher.

Exit orifice 60 is provided in each osmotic dosage form. Exit 60 may encompass one or more exit orifices. Exit 60 cooperates with the drug composition(s) within the dosage form for the uniform release of drug from the dosage form. The exit can be provided during the manufacture of the dosage form or during drug delivery by the dosage form in a fluid environment of use.

Exit 60 may include an orifice that is formed or formable from a substance or polymer that erodes, dissolves or is leached from the outer wall to thereby form an exit orifice. The substance or polymer may include, for example, an erodible poly(glycolic) acid or poly(lactic) acid in the semi-permeable wall; a gelatinous filament; a water-removable poly(vinyl alcohol); a leachable compound, such as a fluid removable pore-former selected from the group consisting of inorganic and organic salt, oxide, carbohydrate, and the like.

The exit 60, or a plurality of exits, can alternatively be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

Exit 60 can have any shape, such as round, triangular, square, oval, elliptical, and the like, for the uniform metered dose release of a drug from the dosage form.

When more than one exit orifice is present in the dosage form, the exits may be present in spaced-apart relation on one or more surfaces of the dosage form, provided that the exit orifices are situated such that they expose drug composition to the external environment.

The drug compositions of the present invention may be prepared according to known methods, for example as a granulation, as a dry blend, as a co-precipitate, as a roller compacted blend, and the like. Preferably, the drug composition is prepared as a granulation.

A variety of processing techniques can be used to promote uniformity of mixing in drug composition 30. In one method, the low solubility and/or low dissolution rate free acid pharmaceutical agent and its pharmaceutically acceptable salt are each micronized to a nominal particle size of less than about 200 microns, preferably, to a nominal particle size of less than about 100 microns, more preferably, to a nominal particle size of less than about 50 microns. Standard micronization processes such as jet milling, cryogrinding, bead milling, and the like, may be used.

Alternatively, the low solubility and/or low dissolution rate free acid pharmaceutical agent and its pharmaceutically acceptable salt may be dissolved in a common solvent to produce mixing at the molecular level and co-dried to a uniform mass. The resulting mass may be ground and sieved to a free-flowing powder. The resulting free-flowing powder may be further, optionally, granulated with wet mass sieving or fluid bed granulation with any optional structural polymer to form a drug composition (in the form of a granulation) of the present invention.

Alternatively still, low solubility and/or low dissolution rate free acid pharmaceutical agent and its pharmaceutically acceptable salt may be melted together at elevated temperature to mix the drug in solubilizing agent, preferably surfactant, and then congealed to room temperature. The resulting solid may be ground, sized, and optionally, further granulated with structural polymer.

In yet another manufacturing process, low solubility and/or low dissolution rate free acid pharmaceutical agent and its pharmaceutically acceptable salt may be dissolved in a common solvent or blend of solvents and spray dried to form a co-precipitate that is then further, optionally incorporated with structural polymer by standard granulation processing by fluid bed processing or wet mass sieving.

In yet another manufacturing process, low solubility and/or low dissolution rate free acid pharmaceutical agent and its pharmaceutically acceptable salt may be dissolved in a common solvent or blend of solvents which pharmaceutical agent/surfactant solution is then sprayed onto the optional structural polymer directly in a fluid bed granulation process.

The drug composition of the present invention may then be formulated into the dosage forms of the present invention. Drug composition 30 within the dosage form is preferably formed by compression of the low solubility and/or low dissolution rate free acid pharmaceutical agent and/or its pharmaceutically acceptable salt and if present, the structural polymer 32. For the preparation of osmotic dosage forms, one or more drug compositions are compressed in a stacked orientation, with a push layer prepared and incorporated into the dosage form in contacting relation to at least one of the drug compositions.

Each drug composition is prepared by mixing the low solubility and/or low dissolution rate free acid pharmaceutical agent optionally with its pharmaceutically acceptable salt and optionally any additional components (e.g. structural polymer 32) into a uniform mixture.

Alternatively, the drug composition may be formed from particles by comminution that produces the size of the low solubility and/or low dissolution rate free acid pharmaceutical agent and/or its pharmaceutically acceptable salt and the size of any accompanying polymers used in the fabrication of the drug composition, typically as a core containing the compound. Means for producing such particles include, but are not limited to, granulation, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller mill, a hammer mill, an attrition mill, a chaser mill, a ball mill, a vibrating ball mill, an impact pulverizer mill, a centrifugal pulverizer, a coarse crusher, a fine crusher, and the like. The size of the particle(s) can be ascertained by screening, including a grizzly screen, a flat screen, a vibrating screen, a revolving screen, a shaking screen, an oscillating screen, a reciprocating screen and the like. The processes and equipment for preparing drug and/or carrier particles are disclosed in Remington's Pharmaceutical Sciences, 18th Ed., pp. 1615-1632 (1990); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

Exemplary solvents suitable for manufacturing drug compositions and/or the push layer for the dosage form comprise aqueous or inert organic solvents that do not adversely harm the materials used in the system. Such solvents include, but are not limited to, members selected from the group consisting of aqueous solvents, alcohols, ketones, esters, ethers, aliphatic hydrocarbons, halogenated solvents, cycloaliphatics, aromatics, heterocyclic solvents and mixtures thereof. Suitable examples of solvents include, but are not limited to, acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

Push layer 40 may be similarly prepared according to known methods, for example according to the processes described above, by mixing the appropriate ingredients under appropriate conditions (e.g. osmoagent, omsopolymer, etc.).

Semi-permeable wall 20 may be similarly prepared according to known methods, for example by pan coating, by mixing the appropriate ingredients and applying the resulting mixture to dosage form.

Dosage form components (e.g. drug composition(s), push layer, semi-permeable wall, exit orifice, etc.) may be combined to form the dosage forms of the present invention according to standard techniques known in the art. More specifically, the dosage form core, comprising one or more drug compositions, and when present the push layer, is prepared first, preferably by compression. The semi-permeable wall is then coated onto the core and one or more exit orifices are provided through the semi-permeable wall to expose one or more drug compositions to the external environment.

For example, the dosage form may be manufactured by the wet granulation technique. In the wet granulation technique, the drug, optional structural polymer and solubilizing agent, preferably surfactant, are blended using an organic solvent, such as denatured anhydrous ethanol, as the granulation fluid. Any additional excipients can then be dissolved in a portion of the granulation fluid, such as the solvent described above, and this latter prepared solution is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven. The dried granules are then sized. Next, magnesium stearate, or another suitable lubricant, is added to the drug granulation, and the granulation is put into milling jars and mixed on a jar mill for up to 10 minutes. The composition is pressed into a layer, for example, in a Manesty® press or a Korsch LCT press.

For a bi-layered core (i.e. a dosage form which comprises a drug composition and a push layer), the drug composition is pressed and a similarly prepared granulation of the push layer is pressed against the drug composition. This intermediate compression typically takes place under a force of about 50-100 newtons. Final stage compression typically takes place at a force of 3500 newtons or greater, often 3500-5000 newtons.

Wherein the core comprises two or more drug compositions and a push layer, each drug composition, prepared as described above is individually compressed. The push layer is then pressed against at least one of the drug compositions, in an intermediate compression step as described above. Final compression of the multi-layer core is then applied as described above.

Single, bi-layer or multi-layer compressed cores are then fed to a dry coater press, e.g., Kilian® Dry Coater press, and subsequently coated with the semi-permeable wall materials, according to known methods.

In another process of manufacture the drug and other ingredients comprising the drug composition are blended and pressed into a solid layer. The layer possesses dimensions that correspond to the internal dimensions of the area the layer is to occupy in the dosage form, and it also possesses dimensions corresponding to the push layer, if included, for forming a contacting arrangement therewith. The drug and other ingredients can also be blended with a solvent and mixed into a solid or semisolid form by conventional methods, such as ball milling, calendering, stirring or roll milling, and then pressed into a preselected shape. Next, if included, the push layer components are placed in contact with the drug composition in a like manner. The layering of the drug composition(s) and the push layer can be fabricated by conventional two-layer press techniques. The compressed cores may then be coated with the semi-permeable wall material, according to known methods.

Another manufacturing process that can be used comprises blending the powdered ingredients for each layer in a fluid bed granulator. After the powdered ingredients are dry blended in the granulator, a granulating fluid, for example, poly(vinylpyrrolidone) in water, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Pan coating may be conveniently used to provide semi-permeable wall 20 of the completed osmotic dosage forms. In the pan coating system, the wall-forming composition (comprising the semi-permeable polymer and optional, additional materials) is deposited by successive spraying of the appropriate wall composition onto the compressed single, bi-layered or multi-layered core (which ore comprises the drug layer(s) and, where present, the push layer), accompanied by tumbling in a rotating pan. A pan coater is often used because of its availability at commercial scale.

Other known coating techniques may alternatively be used for coating the compressed core. For example, semi-permeable wall 20 of the dosage form may be formed in one technique using the air-suspension procedure. This procedure consists of suspending and tumbling the compressed single, bi-layer or multi-layer core in a current of warmed air and the semi-permeable wall forming composition, until the semi-permeable wall is applied to the core. The air-suspension procedure is well suited for independently forming the semi-permeable wall of the dosage form. The air-suspension procedure is described in U.S. Pat. No. 2,799,241; in J. Am. Pharm. Assoc., Vol. 48, pp. 451459 (1959); and, ibid., Vol. 49, pp. 82-84 (1960). The dosage form may alternatively be coated with a Wurster® air-suspension coater using, for example, methylene dichloride methanol as a cosolvent for the wall forming material. An Aeromatic® air-suspension coater may alternatively be used employing a suitable co-solvent.

Once coated, semi-permeable wall 20 is dried in a forced-air oven or in a temperature and humidity controlled oven to free the dosage form of any solvent(s) used in the manufacturing. Drying conditions are conventionally chosen on the basis of available equipment, ambient conditions, solvents, coatings, coating thickness, and the like.

Preferably, the drug compositions, the push layer and/or the dosage forms are dried to remove volatile organic and inorganic solvents to levels that are pharmaceutically acceptable and/or optimal for manufacturing. More preferably, the drug compositions, the push layer and/or the dosage forms are dried to less than about 10% moisture, more preferably still, to less than about 5% moisture, most preferably less than about 3% moisture.

One or more exit orifices are provided according to known methods, for example by drilling, in the drug composition end of the dosage form. Alternatively, one or more exit orifices may be provided in the drug composition end of the dosage form by erosion or leaching.

The dosage form can therefore be constructed with one or more exits in spaced-apart relation on one or more surfaces of the dosage form.

Drilling, including mechanical and laser drilling, through the semi-permeable wall can be used to form the exit orifice. Such exits and equipment for forming such exits are disclosed in U.S. Pat. No. 3,916,899, by Theeuwes and Higuchi and in U.S. Pat. No. 4,088,864, by Theeuwes, et al.

Leachable or erodable exit orifices may be formed or formable from a substance or polymer that erodes, dissolves or is leached from the outer semi-permeable (outer) wall to thereby form an exit orifice. The substance or polymer may include for example, an erodible poly(glycolic)acid or poly(lactic)acid in the semi-permeable wall, a gelatinous filament, a water removable poly(vinyl)alcohol, a leachable compound such as a fluid removable pore former, for example an inorganic or organic salt, oxide or carbohydrate. The exit or plurality of exits can be formed by leaching a member selected from the group consisting of sorbitol, lactose, fructose, glucose, mannose, galactose, talose, sodium chloride, potassium chloride, sodium citrate and mannitol to provide a uniform release dimensioned pore exit orifice. The exit can have any shape, such as, round, triangular, square, elliptical, and the like.

The dosage form may be further, optionally coated with additional water soluble overcoats, which may be colored (e.g., OPADRY colored coatings) or clear (e.g., OPADRY Clear).

The dosage form may further, optionally comprise a smoothing coat, which smoothing coat is applied to the compressed drug core, according to known methods, prior to the application of the semi-permeable wall. Suitable examples of formulations and components which may used in the smoothing coat include, but are not limited to, hydroxypropylcellulose, hydroxyethylcellulose, methylcellulose, hydroxypropyl methylcellulose, and the like. The coating may further optionally contain polyethylene glycol of 400 to 6000 molecular weight, polyvinyl pyrrolidone of 2500 to 1,000,000 molecular weight, and the like.

The dosage forms of the present invention provide controlled release of low solubility and/or low dissolution rate free acid pharmaceutical agent and/or its pharmaceutically acceptable salt over a prolonged period of time, preferably, for greater than about 1 hour, more preferably, for at least about 4 hours, more preferably still, for at least about 8 hours, more preferably, for at least about 10 hours, more preferably still, for at least about 14 hours, more preferably still, for at least 18 hours, more preferably still, for at least 20 hours, more preferably still for at least 22 hours, more preferably still for up to about 24 hours. Preferably, the dosage forms of the present invention provide controlled release of low solubility and/or low dissolution rate free acid pharmaceutical agent and/or its pharmaceutically acceptable salt for about 2 to about 24 hours, more preferably, for about 4 to about 24 hours.

In an embodiment of the present invention, the release of low solubility and/or low dissolution rate free acid pharmaceutical agent and/or its pharmaceutically acceptable salt from the dosage forms of the present invention provides efficacious therapy for about 24 hours. In another embodiment of the present invention, the dosage form releases low solubility and/or low dissolution rate free acid pharmaceutical agent and/or its pharmaceutically acceptable salt for about 16 to about 24 hours after administration.

In an embodiment of the present invention, the dosage form comprises an optional immediate release drug overcoat which provides immediate drug delivery (i.e. within less than about 1 hour after administration) and controlled drug delivery continuing thereafter until the dosage form ceases to release low solubility and/or low dissolution rate free acid pharmaceutical agent, preferably, at least about 8 hours, more preferably, about 12 hours, more preferably still, about 16 hours, more preferably still about 18 hours, more preferably still, about 22 hours, more preferably still, about 24 hours.

Representative dosage forms of the present invention exhibit T₇₀ values of greater than about 8 hours, preferably, greater than about 10 hours, more preferably, greater than about 12 hours, more preferably still, greater than about 16 hours, and release low solubility and/or low dissolution rate free acid pharmaceutical agent and/or its pharmaceutically acceptable salt for a continuous period of time of more than about 12 hours, more preferably, for more than about 16 hours, more preferably still, for about 24 hours.

Within about 2 hours following administration, representative dosage forms of the present invention release low solubility and/or low dissolution rate free acid pharmaceutical agent and/or its pharmaceutically acceptable salt at a substantially zero order rate of release or at a substantially ascending rate of release, depending upon the composition of drug composition(s) and push layers. Preferably, low solubility and/or low dissolution rate free acid pharmaceutical agent and/or its pharmaceutically acceptable salt release continues for a prolonged period of time. Following the prolonged period of delivery, low solubility and/or low dissolution rate free acid pharmaceutical agent and/or its pharmaceutically acceptable salt continues to be delivered for several more hours until the dosage form is spent or expelled from the GI tract.

In a bi-layer embodiment of once-a-day dosage forms in accord with the present invention, the dosage forms have a T₇₀ of about 15 hours to about 18 hours, preferably, about 17 hours, and provided release of low solubility and/or low dissolution rate free acid pharmaceutical agent and/or its pharmaceutically acceptable salt for a continuous period of time, preferably, for at least about 24 hours. Preferably, the dosage form releases low solubility and/or low dissolution rate free acid pharmaceutical agent and/or its pharmaceutically acceptable salt with a substantially zero order rate of release.

In a tri-layer embodiment of the present invention, the dosage form of the present invention comprises two drug compositions and a push layer, wherein the amount and/or concentration of low solubility and/or low dissolution rate free acid pharmaceutical agent and/or its pharmaceutically acceptable salt in the first drug composition is less than the amount and/or concentration of low solubility and/or low dissolution rate free acid pharmaceutical agent and/or its pharmaceutically acceptable salt in the second drug composition. Representative tri-layer dosage forms of the present invention exhibit T₇₀ values of greater than about 8 hours, preferably, greater than about 12 hours, more preferably, greater than about 14 hours, and release low solubility and/or low dissolution rate free acid pharmaceutical agent and/or its pharmaceutically acceptable salt for a continuous period of time of more than about 16 hours, preferably for about 24 hours. Preferably, the dosage form releases low solubility and/or low dissolution rate free acid pharmaceutical agent and/or its pharmaceutically acceptable salt with a substantially ascending rate of release.

In an embodiment of the present invention, the dosage forms of the present invention release the low solubility and/or low dissolution rate free acid pharmaceutical agent and/or its pharmaceutically acceptable salt at various rates of release between about 1%/hr and about 12%/hr over a prolonged period of time.

In an embodiment of the present invention, the dosage forms release low solubility and/or low dissolution rate free acid pharmaceutical agent and/or its pharmaceutically acceptable salt with a substantially zero order rate of release. In another embodiment of the present invention, the dosage forms release low solubility and/or low dissolution rate free acid pharmaceutical agent and/or its pharmaceutically acceptable salt with a substantially ascending rate of release. In yet another embodiment of the present invention, the dosage forms release low solubility and/or low dissolution rate free acid pharmaceutical agent and/or its pharmaceutically acceptable salt with a release rate which results in a substantially ascending drug plasma concentration.

The present invention is further directed to a method of treatment comprising administering any of the drug compositions or dosage forms of the present invention, to a patient in need thereof. Said drug compositions and/or dosage forms comprise low solubility and/or low dissolution rate free acid pharmaceutical agent and/or its pharmaceutically acceptable salt in the range of from about 1 mg to about 750 mg.

The method, in one embodiment, comprises administering orally to a patient in need thereof, a low solubility and/or low dissolution rate free acid pharmaceutical agent and/or its pharmaceutically acceptable salt administered from a dosage form comprising the desired amount of said low solubility and/or low dissolution rate free acid pharmaceutical agent and/or its pharmaceutically acceptable salt.

The present invention further provides methods for administering low solubility and/or low dissolution rate free acid pharmaceutical agent and/or its pharmaceutically acceptable salt to a patient, and methods for producing a desired drug plasma concentration of said low solubility and/or low dissolution rate free acid pharmaceutical agent and/or its pharmaceutically acceptable salt. In an embodiment of the present invention is a method for administering orally to a patient in need thereof, a dosage form that administers at a controlled rate, over a continuous period of time up to about 24 hours, low solubility and/or low dissolution rate free acid pharmaceutical agent and/or its pharmaceutically acceptable salt for its intended therapy. In another embodiment of the present invention, the method comprises administering orally to a patient in need thereof, a therapeutic dose of low solubility and/or low dissolution rate free acid pharmaceutical agent and/or its pharmaceutically acceptable salt from a single dosage form that administers the low solubility and/or low dissolution rate free acid pharmaceutical agent and/or its pharmaceutically acceptable salt over about 24 hours.

The present invention is further directed to a method of treatment comprising administering to a patient in need thereof, an-oral controlled release dosage form of a low solubility and/or low dissolution rate free acid pharmaceutical agent and/or its pharmaceutically acceptable salt wherein the pharmaceutical agent is released from the dosage form in a substantially zero order rate of release.

The present invention is further directed to a method of treating comprising administering to a patient in need thereof, an oral controlled release dosage form of a low solubility and/or low dissolution rate free acid pharmaceutical agent and/or its pharmaceutically acceptable salt wherein the low solubility and/or low dissolution rate free acid pharmaceutical agent and/or its pharmaceutically acceptable salt is released from the dosage form in a substantially ascending rate of release.

The present invention is further directed to a method of treating comprising administering to a patient in need thereof, an oral controlled release dosage form of a low solubility and/or low dissolution rate free acid pharmaceutical agent and/or its pharmaceutically acceptable salt wherein the pharmaceutical agent is released from the dosage form at a rate which results in a substantially ascending drug plasma concentration.

The low solubility and/or low dissolution rate free acid pharmaceutical agent and/or its pharmaceutically acceptable salt that can be delivered according to the invention includes inorganic and organic compounds, including, without limitation, drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autocold systems, the alimentary and excretory systems, the histamine system, and the central nervous system. Suitable agents may be selected from, for example, proteins, enzymes, hormones, polynucleotides, nucleoproteins, polysaccharides, glycoproteins, M. lipoproteins, polypeptides, steroids, hypnotics and sedatives, psychic energizers, tranquilizers, anticonvulsants, muscle relaxants, antiparkinson agents, analgesics, anti-inflammatories, local anesthetics, muscle contractants, antimicrobials, antimalarials, hormonal agents including contraceptives, sympathomimetrics, polypeptides and proteins capable of eliciting physiological effects, diuretics, lipid regulating agents, antiandrogenic agents, antiparasitics, neoplastics, antineoplastics, hypoglycemics, nutritional agents and supplements, growth supplements, fats, ophthalmics, antienteritis agents, electrolytes and diagnostic agents. The present invention is further directed to a method of treating a disorder selected from the group consisting of epilepsy, migraine, glaucoma and other ocular disorders (including diabetic retinopathy), essential tremor, restless limb syndrome, obesity, weight loss, Type II Diabetes Mellitus, Syndrome X, impaired oral glucose tolerance, diabetic skin lesions, cluster headaches, neuralgia, neuropathic pain (including diabetic neuropathy), elevated blood glucose levels, elevated blood pressure, elevated lipids, bipolar disorder, dementia, depression, psychosis, mania, anxiety, schizophrenia, OCD, PTSD, ADHD, impulse control disorders (including bulimia, binge eating, substance abuse, etc.), ALS, asthma, autism, autoimmune disorders (including psoriasis, rheumatoid arthritis, etc.), chronic neurodegenerative disorders, acute neurodegeneration, sleep apnea and other sleep disorders or for promoting wound healing, comprising administering to a patient in need thereof, any of the drug compositions or dosage forms of the present invention.

The following examples are illustrative of the present invention and should not be considered as limiting the scope of the invention in any way, as these examples and other equivalents thereof will become apparent to those versed in the art in light of the present disclosure, drawings and accompanying claims.

### EXAMPLE 1

### Zero Order Release Rate Osmotic Dosage Form of Topiramate

A dosage form adapted, designed and shaped as an osmotic drug delivery device was manufactured as follows: 5 g of topiramate, 11.5 g of topiramate monosodium trihydrate, 29.5 g of polyethylene oxide with average molecular weight of 200,000 and 2.5 g of polyvinylpyrrolidone identified as K29-32 having an average molecular weight of 40,000 (Povidone K29-32) was added to a glass jar. Next, the dry materials were mixed for approximately 30 seconds. Then, approximately 20 ml of denatured anhydrous alcohol was slowly added to the blended materials with continuous mixing for approximately 2 minutes. Next, the freshly prepared wet granulation was allowed to dry at room temperature for approximately 18 hours, and passed through a 16-mesh screen. Next, the granulation was transferred to an appropriate container and lubricated with 1 g of stearic acid and 0.5 g of magnesium stearate.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the drug composition and the push composition were compressed into bilayer tablets on the Carver Tablet Press. First, 278 mg of the topiramate composition was added to the die cavity and pre-compressed, then, 185 mg of the push composition was added and the layers were pressed under a pressure head of approximately ½ a metric ton into a 15/64″ (0.595 cm) diameter bilayer longitudinal arrangement.

The bilayered arrangements were coated with a subcoat laminate. The wall forming composition comprised approximately 70% hydroxypropyl cellulose identified as EF, having an average molecular weight of 80,000 and 30% of polyvinylpyrrolidone identified as K29-32 having an average molecular weight of 40,000. The wall-forming composition was dissolved in anhydrous ethyl alcohol, to make an approximately 8% solids solution. The wall-forming composition was sprayed onto and around the bilayered arrangements in a pan coater until approximately 20 mg of laminate was applied to each tablet.

The bilayered subcoated cores were coated with a semi-permeable wall. The wall forming composition comprised approximately 99% cellulose acetate 398-10 having an acetyl content of approximately 39.8% and 1% polyethylene glycol with a 3,350 viscosity-average molecular weight. The wall-forming composition was dissolved in an acetone:water (95:5 wt:wt) co-solvent to make a 5% solids solution. The wall-forming composition was sprayed onto and around the bilayered arrangements in a pan coater until approximately 45 mg of membrane was applied to each tablet.

Next, a 45 mil (1.1 mm) exit passageway was drilled through the semi-permeable wall to connect the drug layer with the exterior of the dosage system. The residual solvent was removed by drying for 72 hours at 40 Deg. C. and ambient humidity.

The dosage form produced by this manufacture was designed to deliver 80 mg of topiramate free acid equivalent in a controlled delivery pattern from the drug-containing core. The core contained approximately 10% topiramate, 23% topiramate monosodium trihydrate, 59% polyethylene oxide possessing a 200,000 molecular weight, 5% of polyvinylpyrrolidone (Povidone K29-32), 1% of magnesium stearate and 2% stearic acid. The push composition comprised approximately 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The subcoat was comprised of approximately 70% hydroxypropyl cellulose identified as EF, having an average molecular weight of 80,000 and 30% of polyvinylpyrrolidone identified as K29-32 having an average molecular weight of 40,000. The semi permeable wall was comprised of approximately 99% cellulose acetate of 39.8% acetyl content and 1% polyethylene glycol. The dosage form comprised one passageway, 45 mils (1.1 mm) on the center of the drug side.

### EXAMPLE 2

### Dissolution Test—Zero Order

Dosage forms produced according to Example 1 were tested to determine the topiramate release rate by high performance liquid chromatography (HPLC). The amount of topiramate in sample solutions was analyzed by HPLC using reverse phase C8 column with a refractive index detector. Quantitation was performed by linear regression analysis of the peak areas from a standard curve containing at least seven standard points.

Supplies used were: Chemicals and Reagents: Acetonitrile (ACN, HPLC grade), Methanol (MeOH, HPLC grade), Alza Milli-Q (18.2 MW-cm) or deionized water (D.I. H2O), topiramate reference standard, (topiramate of known purity, obtainable from commercial source); Glassware and Supplies: Class A volumetric flasks and pipettes, 50 mL calibrated test tubes, Screw capped test tubes, HPLC vials compatible with autosampler used, and Prong sample holder (0.44 inch size prong). Equipment used: Balance—Five-place analytical (reading to 0.01 mg), Bath—USP Type VII Apparatus, Centrifuge—IEC (CR-600) or equivalent, HPLC—Pump=Waters 515 or equivalent; Detector=Shimadzu Refractive Index (RI) Detector RID-10A or Waters 2414 RID; Injector=Waters 717 Auto sampler, Column=Waters Symmetry C8, 4.6×150 mm, 5.0 mm, Guard Column=MetaChem Inertsil C8, 5.0 mm, Column Heater=Eppendorf CH-30 with TC-50 temperature controller, Data Reduction=TotalChrom version 6.2.0.0. NOTE: Manufacturer and model names are provided as guidelines and may be substituted with qualified equivalent equipment. All applicable equipment calibrations should be verified as being current.

Reagent preparation: Four liters of mobile phase were prepared as follows: 2200 mL D.I.H2O, 1000 mL Methanol, and 800 mL of Acetonitrile were added to an appropriate container. The contents were mixed well and degassed prior to use. Two liters of Reagent 1 were prepared as follows: 1600 mL D.I. H2O and 400 mL Acetonitrile were added to an appropriate container and mixed well. Two liters of Reagent 2 were prepared as follows: 1000 mL Methanol and 1000 mL Acetonitrile were added to an appropriate container and mixed well.

Next, a topiramate standard stock solution was made. Approximately ˜130 mg of topiramate reference standard were weighed into a 200 mL volumetric flask to make a stock solution with concentration of about 650 μg/mL topiramate. Approximately 100 mL of reagent 1 were added into the flask and sonicated until topiramate dissolved. The flask was cooled down to ambient temperature. The flask was brought to volume with reagent 1 and mixed well.

Next, a topiramate QC Stock Standard Solution with concentration of about 200 pg/mL topiramate was made up. Approximately ˜40 mg of topiramate reference standard was weighed into a 200 mL volumetric flask to make QC stock solution. Approximately 100 mL of regent 1 was added to the flask and the contents were sonicated until dissolved. The flask contents were cooled down to ambient temperature, brought to volume with reagent 1 and mixed well.

Topiramate Working Standards were made up as follows: serial dilutions of the topiramate stock standard solution were made into appropriate volumetric flasks using reagent 1 as the diluent. (See Table 1 for recommended dilution scheme and analysis for the calibration curve.)

TABLE 1
Suggested Standard Dilution Scheme for
Release Rate and Residual Drug Analyses

| | | | | Flask | Approximate |
|---|---|---|---|---|---|
| Standard | Amount/Pipette | | | Volume | Concentration |
| (ID) | Volume | | Component | (mL) | (μg/mL) |
| Std Stock | 130 | mg | Topiramate | 200 | 650 |
| Std-7 | | | | | |
| Std-6 | 25 | mL | Stock Std | 50 | 325 |
| Std 5 | 15 | mL | Stock Std | 50 | 195 |
| Std-4 | 10 | mL | Stock Std | 50 | 130 |
| Std-3 | 7 | mL | Stock Std | 100 | 45.5 |
| Std-2 | 8 | mL | Std-5 | 50 | 31.2 |
| Std-1 | 3 | mL | Std-4 | 50 | 7.8 |
| QC Std | 40 | mg | Topiramate | 200 | 200 |

* Calculation was based on 100% purity.

The stock standard, working standards and QC standard for topiramate were considered stable for 30 days at ambient condition.

Next, each dosage form that was produced according to Example 1 and that was to be tested was weighed and the weight recorded. Each dosage form was placed in a prong sample holder. The prong sample holder was attached to the USP VII bath indexer that operated at a vertical reciprocating amplitude of about 2-3 cm, and a frequency of about 30 cycles per minute. The dosage forms were released into 50 mL calibrated test tubes containing 50 mL D.I.H2O at 37.0° C.±0.5° C. such that the dosage forms were continuously immersed. Test tube solutions were pre-equilibrated in a constant temperature water bath controlled to 37.0° C.±0.5° C.

At the end of each two hour test interval, the dosage forms were transferred to the next row of test tubes containing fresh D.I.H2O. After release, the tubes were removed from the bath and allowed to cool to ambient temperature. The release solution in each tube was brought up to the 50 ml mark with D.I. H2O, and thoroughly mixed 30 times using an inert stirring rod fitted with a disk perpendicular to the rod. Cloudy solutions were centrifuged for about 10 minutes at approximately 2000 rpm or until solution is clear. An aliquot was transferred to an HPLC vial. NOTE: Release rate sample solution were considered stable for 7 days at ambient condition.

The HPLC operating parameters were set as follows: Flow Rate=1.3 mL/min, Injection Volume=5 mL, Column Temperature=40° C., Run time=5.5 min (nominal), Detector temperature=40° C., AUX:1 (nominal, for Shimadzu RI detectors only), Sensitivity=64 (nominal, for Waters 2414 RI detectors only). A System Suitability Analysis was performed by equilibrating the HPLC system until a steady baseline is obtained. Five replicate injections were performed of a medium range standard of topiramate. The system was considered suitable for analysis if the following minimum chromatographic performance requirements were met:
Capacity Factor (k′): ≧1.5
Tailing Factor (T): 0.5≦T≦2.5
Response Variation (% RSD): ≦2%
Retention Time Variation (% RSD): ≦5%
Theoretical Plate: Report Result

Adjustments were made to run time or columns were replaced as necessary to obtain optimum performance. The mobile phase was thoroughly degassed prior to HPLC run. If a distorted peak or increasing back pressure was observed, the column was cleaned by flushing 50:50 MeOH:Acetonitrile or THF. Columns were reversed on occasion to expedite the cleaning process. The flow cell for the RI detector was cleaned periodically to avoid unstable baseline. RI detector was cleaned by flushing 50:50 MeOH:Acetonitrile through the flow cell. Refer to the manufacturer's RI detector manual for detailed cleaning.

Samples were analyzed by injecting mobile phase and solvent blanks to ensure the sum of all detected peaks within ±5% of the retention time of the topiramate peak should be ≦1% (area %) of the mid-range/target concentration level for release rate and residual drug analyses. A standard calibration curve was established by injecting working standards to bracket the expected sample concentration range. The correlation coefficient (r²) was targeted as ≧0.990. The % recovery of the standards should be within ±3% of the theoretical concentrations. For the lowest standard, the % recovery of ±5% was considered acceptable. Weighting factor of 1/x must be applied to the regression line of release rate and residual drug curves to enhance the accuracy of the low end standard concentrations.

A QC standard was injected prior to any sample analysis. The % recovery for the QC standard should be within ±3% of the theoretical concentration. QC standard(s) or mid-range check standard(s) were injected periodically during and at the end of the analysis to check the system performance. The % recovery should be within ±3% of the theoretical concentrations for all QC standards. The % RSD was calculated if there were more than two QC standard(s) or mid-range check standard(s) injections. The % RSD should be £2%. for all types of analyses. If there are only two QC standard or mid-range check standard injections, the % difference should be ≦3%.

Next, a calibration curve of peak area response versus concentration of working standards was constructed. The concentration of topiramate in sample solutions was determined from the linear regression analysis of the calibration curve. For release rate samples, the release rate (mg/hr) and the cumulative drug released (mg) or the cumulative % label claim (% LC) of the drug released as needed was calculated.

$$mg drug \text{/} hr = \frac{C \times V}{1000 \times T}$$

where:

C=Concentration of sample by linear regression analysis obtained from the calibration curve in mg/mL

V=Volume of release media in mL (e.g. 50 mL)

T=Time interval in hours

n=Number of sampling points

1000=Conversion factor from mg to mg

dosage=dosage strength of the tablet (e.g. 100 mg)

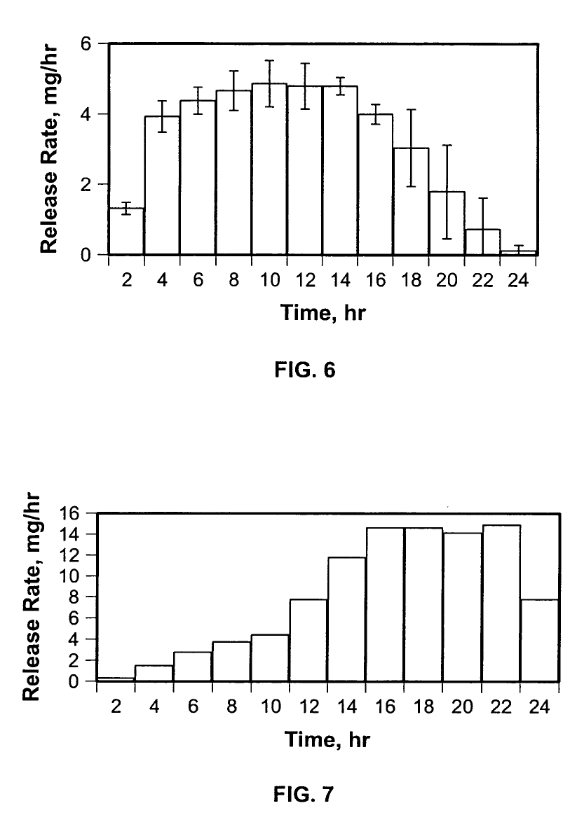

The results were as shown in FIG. 6.

### EXAMPLE 3

### Ascending Release Rate Topiramate Dosage Form

A dosage form adapted, designed and shaped as an osmotic drug delivery device was manufactured as follows: for the first drug layer 5 g of topiramate, 13.4 g of polyethylene oxide with average molecular weight of 200,000 and 1 g of polyvinylpyrrolidone identified as K29-32 having an average molcular weight of 40,000 (Povidone K29-32) ware added to a glass jar. Next, the dry materials were mixed for 30 seconds. Then, approximately 5 ml of denatured anhydrous alcohol was slowly added to the blended materials with continuous mixing for approximately 2 minutes. Next, the freshly prepared wet granulation was allowed to dry at room temperature for approximately 18 hours, and passed through a 16-mesh screen. Next, the granulation was transferred to an appropriate container and lubricated with 0.4 g of stearic acid and 0.2 g of magnesium stearate.

Next, the second drug layer was prepared as follows: 5 g of topiramate, 12 g of topiramate monosodium trihydrate, 1.4 g of polyethylene oxide with average molecular weight of 200,000 and 1 g of polyvinylpyrrolidone (Povidone K29-32) were added to a glass jar. Next, the dry materials were mixed for 30 seconds. Then, approximately 5 ml of denatured anhydrous alcohol was slowly added to the blended materials with continuous mixing for approximately 2 minutes. Next, the freshly prepared wet granulation was allowed to dry at room temperature for approximately 18 hours, and passed through a 16-mesh screen. Next, the granulation was transferred to an appropriate container and lubricated with 0.4 g of stearic acid and 0.2 g of magnesium stearate.

Next, a push composition was prepared as follows: first, a binder solution was prepared. 15.6 kg of polyvinylpyrrolidone identified as K29-32 having an average molecular weight of 40,000 was dissolved in 104.4 kg of water. Then, 24 kg of sodium chloride and 1.2 kg of ferric oxide were sized using a Quadro Comil with a 21-mesh screen. Then, the screened materials and 88.44 kg of Polyethylene oxide (approximately 7,000,000 molecular weight) were added to a fluid bed granulator bowl. The dry materials were fluidized and mixed while 46.2 kg of binder solution was sprayed from 3 nozzles onto the powder. The granulation was dried in the fluid-bed chamber to an acceptable moisture level. The coated granules were sized using a Fluid Air mill with a 7-mesh screen. The granulation was transferred to a tote tumbler, mixed with 15 g of butylated hydroxytoluene and lubricated with 294 g magnesium stearate.

Next, the first drug composition, the second drug layer composition and the push composition were compressed into bilayer tablets on the Carver Tablet Press. First, 240 mg of the first drug layer composition was added to the die cavity and pre-compressed, then, 240 mg of the second drug layer composition was added to the die cavity and pre-compressed, and finally, 360 mg of the push composition was added and the layers were pressed under a pressure head of approximately 0.5 metric ton into a 9/32″ (0.714 cm) diameter bilayer longitudinal arrangement.

The trilayered arrangements were coated with a subcoat laminate. The wall forming composition comprised approximately 95% 2-hydroxyethyl-cellulose ether, having an average viscosity of 200 mPa s and 5% of polyethylene glycol identified as 3350 having an average molecular weight of 3350. The wall-forming composition was dissolved in USP water, to make an approximately 6% solids solution. The wall-forming composition was sprayed onto and around the trilayered arrangements in a pan coater until approximately 20 mg of laminate was applied to each tablet.

The trilayered-subcoated cores were coated with a semi-permeable wall. The wall forming composition comprises approximately 99% cellulose acetate 398-10 having an acetyl content of approximately 39.8% and 1% polyethylene glycol with a 3,350 viscosity-average molecular weight. The wall-forming composition was dissolved in an acetone:water (95:5 wt:wt) co solvent to make a 5% solids solution. The wall-forming composition was sprayed onto and around the bilayered arrangements in a pan coater until approximately 80 mg of membrane was applied to each tablet.

Next, an 83 mil (2.1 mm) exit passageway was drilled through the semi-permeable wall to connect the drug layer with the exterior of the dosage system. The residual solvent was removed by drying for 72 hours at 40 Deg C. and ambient humidity.

The dosage form produced by this manufacture was designed to deliver 238 mg of topiramate free acid equivalent in a controlled delivery pattern from the drug-containing core. The drug-containing layers contained approximately 25% topiramate, 30% topiramate monosodium trihydrate, 37% polyethylene oxide possessing a 200,000 molecular weight, 5% of polyvinylpyrrolidone (Povidone K29-32), 1% of magnesium stearate and 2% stearic acid. The push composition comprised approximately 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The subcoat comprised approximately 95% 2-hydroxyethyl-cellulose ether, having an average viscosity of 200-mPa s and 5% of polyethylene glycol identified as 3350. The semi permeable wall comprised approximately 99% cellulose acetate of 39.8% acetyl content and 1% polyethylene glycol. The dosage form comprised one passageway, 83 mils (2.1 mm) on the center of the drug side.

### EXAMPLE 4

### Dissolution Test—Ascending Profile

Dosage forms produced according to Example 3 were tested to determine the topiramate release rate by high performance liquid chromatography (HPLC). The amount of topiramate in sample solutions was analyzed by HPLC using reverse phase C8 column with a refractive index detector. Quantitation was performed by linear regression analysis of the peak areas from a standard curve containing at least seven standard points.

Supplies used were: Chemicals and Reagents: Acetonitrile (ACN, HPLC grade), Methanol (MeOH, HPLC grade), Alza Milli-Q (18.2 MW-cm) or deionized water (D.I. H2O), topiramate reference standard (topiramate of known purity, obtainable from commercial source); Glassware and Supplies: Class A volumetric flasks and pipettes, 50 mL calibrated test tubes, Screw capped test tubes, HPLC vials compatible with autosampler used, and Prong sample holder (0.44 inch size prong). Equipment used: Balance—Five-place analytical (reading to 0.01 mg), Bath—USP Type VII Apparatus, Centrifuge—IEC (CR-600) or equivalent, HPLC—Pump=Waters 515 or equivalent; Detector=Shimadzu Refractive Index (RI) Detector RID-10A or Waters 2414 RID; Injector=Waters 717 Auto sampler, Column=Waters Symmetry C8, 4.6×150 mm, 5.0 mm, Guard Column=MetaChem Inertsil C8, 5.0 mm, Column Heater=Eppendorf CH-30 with TC-50 temperature controller, Data Reduction=TotalChrom version 6.2.0.0. NOTE: Manufacturer and model names are provided as guidelines and may be substituted with qualified equivalent equipment. All applicable equipment calibrations should be verified as being current.

Reagent preparation: Four liters of mobile phase were prepared as follows: 2200 mL D.I.H2O, 1000 mL Methanol, and 800 mL of Acetonitrile were added to an appropriate container. The contents were mixed well and degassed prior to use. Two liters of Reagent 1 were prepared as follows: 1600 mL D.I. H2O and 400 mL Acetonitrile were added to an appropriate container and mixed well. Two liters of Reagent 2 were prepared as follows: 1000 mL Methanol and 1000 mL Acetonitrile were added to an appropriate container and mixed well.

Next, a topiramate standard stock solution was made. Approximately ˜130 mg of topiramate reference standard were weighed into a 200 mL volumetric flask to make a stock solution with concentration of about 650 μg/mL topiramate. Approximately 100 mL of reagent 1 were added into the flask and sonicated until topiramate dissolved. The flask was cooled down to ambient temperature. The flask was brought to volume with reagent 1 and mixed well.

Next, a topiramate QC Stock Standard Solution with concentration of about 200 μg/mL topiramate was made up. Approximately ˜40 mg of topiramate reference standard was weighed into a 200 mL volumetric flask to make QC stock solution. Approximately 100 mL of regent 1 was added to the flask and the contents were sonicated until dissolved. The flask contents were cooled down to ambient temperature, brought to volume with reagent 1 and mixed well.

Topiramate Working Standards were made up as follows: serial dilutions of the topiramate stock standard solution were made into appropriate volumetric flasks using reagent 1 as the diluent. (See Table 1 for recommended dilution scheme and analysis for the calibration curve.)

The stock standard, working standards and QC standard for topiramate were considered stable for 30 days at ambient condition.

Next, each dosage form that was produced according to Example 3 and that was to be tested was weighed and the weight recorded. Each dosage form was placed in a prong sample holder. The prong sample holder was attached to the USP VII bath indexer that operated at a vertical reciprocating amplitude of about 2-3 cm, and a frequency of about 30 cycles per minute. The dosage forms were released into 50 mL calibrated test tubes containing 50 mL D.I.H2O at 37.0° C.±0.5° C. such that the dosage forms were continuously immersed. Test tube solutions were pre-equilibrated in a constant temperature water bath controlled to 37.0° C.±0.5° C.

At the end of each 2 hours test interval, the dosage forms were transferred to the next row of test tubes containing fresh D.I.H2O. After release, the tubes were removed from the bath and allowed to cool to ambient temperature. The release solution in each tube was brought up to the 50 ml mark with D.I. H2O, and thoroughly mixed 30 times using an inert stirring rod fitted with a disk perpendicular to the rod. Cloudy solutions were centrifuged for about 10 minutes at approximately 2000 rpm or until solution is clear. An aliquot was transferred to an HPLC vial. NOTE: Release rate sample solution were considered stable for 7 days at ambient condition.

The HPLC operating parameters were set as follows: Flow Rate=1.3 mL/min, Injection Volume=5 mL, Column Temperature=400C, Run time=5.5 min (nominal), Detector temperature=40° C., AUX:1 (nominal, for Shimadzu RI detectors only), Sensitivity=64 (nominal, for Waters 2414 RI detectors only). A System Suitability Analysis was performed by equilibrating the HPLC system until a steady baseline is obtained. Five replicate injections were performed of a medium range standard of topiramate. The system was considered suitable for analysis if the following minimum chromatographic performance requirements were met:
Capacity Factor (k′): ≧1.5
Tailing Factor (T): 0.5≦T≦2.5
Response Variation (% RSD): ≦2%
Retention Time Variation (% RSD): ≦5%
Theoretical Plate: Report Result

Adjustments were made to run time or columns were replaced as necessary to obtain optimum performance. The mobile phase was thoroughly degassed prior to HPLC run. If a distorted peak or increasing back pressure was observed, the column was cleaned by flushing 50:50 MeOH:Acetonitrile or THF. Columns were reversed on occasion to expedite the cleaning process. The flow cell for the RI detector was cleaned periodically to avoid unstable baseline. RI detector was cleaned by flushing 50:50 MeOH:Acetonitrile through the flow cell. Refer to the manufacturer's RI detector manual for detailed cleaning.

Samples were analyzed by injecting mobile phase and solvent blanks to ensure the sum of all detected peaks within ±5% of the retention time of the topiramate peak should be ≦1% (area %) of the mid-range/target concentration level for release rate and residual drug analyses. A standard calibration curve was established by injecting working standards to bracket the expected sample concentration range. The correlation coefficient (r²) was targeted as ≧0.990. The % recovery of the standards should be within ±3% of the theoretical concentrations. For the lowest standard, the % recovery of ±5% was considered acceptable. Weighting factor of 1/x must be applied to the regression line of release rate and residual drug curves to enhance the accuracy of the low end standard concentrations.

A QC standard was injected prior to any sample analysis. The % recovery for the QC standard should be within ±3% of the theoretical concentration. QC standard(s) or mid-range check standard(s) were injected periodically during and at the end of the analysis to check the system performance. The % recovery should be within ±3% of the theoretical concentrations for all QC standards. The % RSD was calculated if there were more than two QC standard(s) or mid-range check standard(s) injections. The % RSD should be £2% for all types of analyses. If there are only two QC standard or mid-range check standard injections, the % difference should be ≦3%.

Next, a calibration curve of peak area response versus concentration of working standards was constructed. The concentration of topiramate in sample solutions was determined from the linear regression analysis of the calibration curve. For release rate samples, the release rate (mg/hr) and the cumulative drug released (mg) or the cumulative % label claim (% LC) of the drug released as needed was calculated.

$$mg drug \text{/} hr = \frac{C \times V}{1000 \times T}$$

where:

C=Concentration of sample by linear regression analysis obtained from the calibration curve in mg/mL

V=Volume of release media in mL (e.g. 50 mL)

T Time interval in hours

n=Number of sampling points

1000=Conversion factor from mg to mg

dosage=dosage strength of the tablet (e.g. 100 mg)

The results were as shown in FIG. 7:

### EXAMPLE 5

### Zero Order Release Rate Osmotic Dosage Form of Valproic Acid/Sodium Valproate

A dosage form adapted, designed and shaped as an osmotic drug delivery device is manufactured as follows: 10.0 g of valproic acid, 23 g of sodium valproate, 59 g of polyethylene oxide with average molecular weight of 200,000 and 5 g of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container and lubricated with 2 g of stearic acid and 1 g of magnesium stearate.

Next, a push composition is prepared as follows: first, a binder solution is prepared. 15.6 kg of polyvinylpyrrolidone identified as K29-32 having an average molecular weight of 40,000 is dissolved in 104.4 kg of water. Then, 24 kg of sodium chloride and 1.2 kg of ferric oxide are sized using a Quadro Comil with a 21-mesh screen. Then, the screened materials and 88.44 kg of Polyethylene oxide (approximately 7,000,000 molecular weight) are added to a fluid bed granulator bowl. The dry materials are fluidized and mixed while 46.2 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 15 g of butylated hydroxytoluene and lubricated with 294 g magnesium stearate.

Next, the drug composition and the push composition are compressed into bilayer tablets on the Carver Tablet Press. First, 278 mg of the valproic acid composition is added to the die cavity and pre-compressed, then, 185 mg of the push composition is added and the layers are pressed under a pressure head of approximately ½ metric ton into a 15/64″ (0.586 cm) diameter bilayer longitudinal arrangement.

The bilayered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000. The wall-forming composition is dissolved in anhydrous ethyl alcohol, to make an 8% solids solution. The wall-forming composition is sprayed onto and around the bilayered arrangements in a pan coater until approximately 20 mg of laminate is applied to each tablet.

The bilayered subcoated cores are coated with a semi-permeable wall. The wall forming composition comprises 99% cellulose acetate having a 39.8% acetyl content and 1% polyethylene glycol comprising a 3,350 viscosity-average molecular weight. The wall-forming composition is dissolved in an acetone:water (95:5 wt:wt) co solvent to make a 5% solids solution. The wall-forming composition is sprayed onto and around the bilayered arrangements in a pan coater until approximately 45 mg of membrane is applied to each tablet.

Next, a 45 mil (1.1 mm) exit passageway is drilled through the semi-permeable wall to connect the drug layer with the exterior of the dosage system. The residual solvent is removed by drying for 72 hours as 40 C and ambient humidity.

The dosage form produced by this manufacture is designed to deliver 83.4 mg of valproic acid (free acid equivalent) in a controlled delivery pattern from the drug-containing core. The core contains 10% valproic acid, 23% sodium valproate, 59% polyethylene oxide possessing a 200,000 molecular weight, 5% of polyvinylpyrrolidone (Povidone K29-32), 1% of magnesium stearate and 2% stearic acid. The push composition is comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The subcoat is comprised of 70% hydroxypropyl cellulose identified as EF, having an average molecular weight of 80,000 and 30% of polyvinylpyrrolidone identified as K29-32 having an average molecular weight of 40,000. The semi permeable wall is comprised of 99% cellulose acetate of 39.8% acetyl content and 1% polyethylene glycol. The dosage form comprises one passageway, 45 mils (1.1 mm) on the center of the drug side.

### EXAMPLE 6

### Zero Order Release Rate Osmotic Dosage Form of Acyclovir/Sodium Acyclovir

A dosage form adapted, designed and shaped as an osmotic drug delivery device is manufactured as follows: 10.0 g of acyclovir, 23 g of sodium acyclovir, 59 g of polyethylene oxide with average molecular weight of 200,000 and 5 g of polyvinylpyrrolidone (Povidone K29-32) are added to a glass jar. Next, the dry materials are mixed for 30 seconds. Then, 20 ml of denatured anhydrous alcohol is slowly added to the blended materials with continuous mixing for approximately 2 minutes. Next, the freshly prepared wet granulation is allowed to dry at room temperature for approximately 18 hours, and passed through a 16-mesh screen. Next, the granulation is transferred to an appropriate container and lubricated with 2 g of stearic acid and 1 g of magnesium stearate.

Next, a push composition is prepared as follows: first, a binder solution is prepared. 15.6 kg of polyvinylpyrrolidone identified as K29-32 having an average molecular weight of 40,000 is dissolved in 104.4 kg of water. Then, 24 kg of sodium chloride and 1.2 kg of ferric oxide are sized using a Quadro Comil with a 21-mesh screen. Then, the screened materials and 88.44 kg of Polyethylene oxide (approximately 7,000,000 molecular weight) are added to a fluid bed granulator bowl. The dry materials are fluidized and mixed while 46.2 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 15 g of butylated hydroxytoluene and lubricated with 294 g magnesium stearate.

Next, the drug composition and the push composition are compressed into bilayer tablets on the Carver Tablet Press. First, 278 mg of the acyclovir composition is added to the die cavity and pre-compressed, then, 185 mg of the push composition is added and the layers are pressed under a pressure head of approximately ½ metric ton into a 15/64″ (0.586 cm) diameter bilayer longitudinal arrangement.

The bilayered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000. The wall-forming composition is dissolved in anhydrous ethyl alcohol, to make an 8% solids solution. The wall-forming composition is sprayed onto and around the bilayered arrangements in a pan coater until approximately 20 mg of laminate is applied to each tablet.

The bilayered subcoated cores are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co solvent to make a 5% solids solution. The wall-forming composition is sprayed onto and around the bilayered arrangements in a pan coater until approximately 45 mg of membrane is applied to each tablet.

Next, a 45 mil (1.1 mm) exit passageway is drilled through the semi-permeable wall to connect the drug layer with the exterior of the dosage system. The residual solvent is removed by drying for 72 hours as 40 C and ambient humidity.

The dosage form produced by this manufacture is designed to deliver 86.2 mg of acyclovir (free acid equivalent) in a controlled delivery pattern from the drug-containing core. The core contains 10% acyclovir, 23% sodium acyclovir, 59% polyethylene oxide possessing a 200,000 molecular weight, 5% of polyvinylpyrrolidone (Povidone K29-32), 1% of magnesium stearate and 2% stearic acid. The push composition is comprised 73.7% polyethylene oxide comprising a 7,000,000 molecular weight, 20% sodium chloride, 5% polyvinylpyrrolidone possessing an average molecular weight of 40,000, 1% ferric oxide, 0.05% butylated hydroxytoluene, and 0.25% magnesium stearate. The subcoat is comprised of 70% hydroxypropyl cellulose identified as EF, having an average molecular weight of 80,000 and 30% of polyvinylpyrrolidone identified as K29-32 having an average molecular weight of 40,000. The semi permeable wall is comprised of 99% cellulose acetate of 39.8% acetyl content and 1% polyethylene glycol. The dosage form comprises one passageway, 45 mils (1.1 mm) on the center of the drug side.

In as much as the foregoing specification comprises disclosed embodiments, it is understood what variations and modifications may be made herein, in accordance with the principles disclosed, without departing from the invention.

1. An osmotic controlled release dosage form comprising a drug composition comprising:
a low solubility and/or low dissolution rate free acid pharmaceutical agent, and
a pharmaceutically acceptable salt thereof.

2. The osmotic controlled release dosage form of claim 1, wherein the drug composition comprises acyclovir, aspirin, azathioprine, cefoxitin, furosemide, ganciclovir, glipizide, ibuprofen, ketoprofen, mefenamic acid, methotrexate, omeprazole, phenobarbitol, topiramate, valproic acid, or combinations thereof.

3. The osmotic controlled release dosage form of claim 2, wherein the drug composition comprises topiramate.

4. The osmotic controlled release dosage form of claim 1, with the proviso that the drug composition is substantially free from solubilizing agents.

5. An osmotic controlled release dosage form comprising
a core comprising a first drug composition, wherein the first drug composition comprises a low solubility and/or low dissolution rate free acid pharmaceutical agent and/or its pharmaceutically acceptable salt;
a semi-permeable wall surrounding the core; and
an exit orifice through the semi-permeable wall for releasing the first drug composition from the dosage form over a prolonged period of time

6. The osmotic controlled release dosage form of claim 5, wherein the weight ratio of low solubility and/or low dissolution rate free acid pharmaceutical agent to its pharmaceutically acceptable salt in the first drug composition is in the range of from about 0.25 to about 2.0

7. The osmotic controlled release dosage form of claim 6, wherein the weight ratio of low solubility and/or low dissolution rate free acid pharmaceutical agent to its pharmaceutically acceptable salt in the first drug composition is in the range of from about 0.3 to about 1.5

8. The osmotic controlled release dosage form of claim 7, wherein the weight ratio of low solubility and/or low dissolution rate free acid pharmaceutical agent to its pharmaceutically acceptable salt in the first drug composition is in the range of from about 0.5 to about 1.0.

9. An osmotic controlled release dosage form comprising
a core comprising
a first drug composition,
a second drug composition and
a push layer,
wherein the first and second drug composition each comprise a low solubility and/or low dissolution rate free acid pharmaceutical agent and/or its pharmaceutically acceptable salt;
a semi-permeable wall surrounding the core; and
an exit orifice through the semi-permeable wall for releasing the first and second drug compositions from the dosage form over a prolonged period of time.

10. The osmotic controlled release dosage form of claim 9, wherein the first drug composition comprises low solubility and/or low dissolution rate free acid pharmaceutical agent and is substantially free from its pharmaceutically acceptable salt, and the second drug composition comprises the pharmaceutically acceptable salt and is substantially free from the low solubility and/or low dissolution rate free acid pharmaceutical agent.

11. The osmotic controlled release dosage form of claim 9, wherein the first drug composition comprises low solubility and/or low dissolution rate free acid pharmaceutical agent and its pharmaceutically acceptable salt in a weight ratio of about 0.5 to about 5.0 acid:salt

12. The osmotic controlled release dosage form of claim 9, wherein the second drug composition comprises the low solubility and/or low dissolution rate free acid pharmaceutical agent and its pharmaceutically acceptable salt in a weight ratio of about 0.15 to about 2.0 acid:salt.
